SEQUENCE LISTING (6 sequences)

SEQ ID NO: 1 - DNA, 26 bp, synthetic construct
  source 1..26: mol_type = other DNA
  misc_feature 1..26: note = D-loop Forward primer
  variation 1: note = Y is C or T
  variation 3: note = Y is C or T
  variation 14: note = Y is C or T
  variation 24: note = Y is C or T
yayttgggggtagytaaagtgaaytg

SEQ ID NO: 2 - DNA, 28 bp, synthetic construct
  source 1..28: mol_type = other DNA
  misc_feature 1..28: note = D-loop Reverse primer
  variation 9: note = R is A or G
tcctacaarcattaattaattaacacac

SEQ ID NO: 3 - DNA, 27 bp, synthetic construct
  source 1..27: mol_type = other DNA
  misc_feature 1..27: note = ND1 gene Forward primer
  variation 7: note = Y is C or T
  variation 19: note = Y is C or T
  variation 23: note = Y is C or T
  variation 14: note = Y is C or T
ataaaayttaaaaytttayagtyagag

SEQ ID NO: 4 - DNA, 25 bp, synthetic construct
  source 1..25: mol_type = other DNA
  misc_feature 1..25: note = ND1 gene Reverse primer
  variation 3: note = R is A or G
  variation 5: note = R is A or G
  variation 9: note = R is A or G
  variation 12: note = R is A or G
  variation 21: note = R is A or G
ttrartttratrctcaccctratca

SEQ ID NO: 5 - DNA, 33 bp, synthetic construct
  source 1..33: mol_type = other DNA
  misc_feature 1..33: note = Illumina forward overhang adapter
tcgtcggcagcgtcagatgtgtataagagacag

SEQ ID NO: 6 - DNA, 34 bp, synthetic construct
  misc_feature 1..34: note = Illumina reverse overhang adapter
  source 1..34: mol_type = other DNA
gtctcgtgggctcggagatgtgtataagagacag

(19) United States
(12) Patent Application Publication
(10) Pub. No.: US 2025/0313893 A1
(43) Pub. Date: Oct. 9, 2025

(54) METHODS OF DETERMINING THE RISK OF DEVELOPING ALZHEIMER'S DISEASE DEMENTIA

(71) Applicant: ADMIT THERAPEUTICS SL, Esplugues Del Llobregat (ES)

(72) Inventors: Marta BARRACHINA CASTILLO, Esplugues Del Llobregat (ES); Jose Luis MOSQUERA MAYO, Esplugues Del Llobregat (ES); Marta BLANCH LOZANO, Esplugues Del Llobregat (ES)

(21) Appl. No.: 18/845,877

(22) PCT Filed: Mar. 10, 2023
(86) PCT No.: PCT/EP2023/056250

§ 371 Date: Sep. 10, 2024

(30) Foreign Application Priority Data
Mar. 11, 2022 (EP) 22382237.0
Nov. 25, 2022 (EP) 22383135.5

(51) Int. Cl. C12Q 1/6883; G06N 20/00; G16B 20/00; G16B 40/20; G16H 50/30

(52) U.S. Cl. CPC C12Q 1/6883; G06N 20/00; G16B 20/00; G16B 40/20; G16H 50/30; C12Q 2600/118; C12Q 2600/154

(57) ABSTRACT

It is provided a method of determining the risk of developing Alzheimer's disease dementia in a subject, comprising: (a) determining in a sample of the subject comprising mitochondrial DNA, the methylation pattern in the D-loop region, and/or in the ND1 gene of the mitochondrial DNA; and (b) combining the methylation pattern of one or more sites determined in step (a), with at least one clinical variable of the subject, wherein said combining is performed using a classification model for determining a risk score which correlates to the risk of developing Alzheimer's disease dementia in the subject. A classification model, oligonucleotides, and kits to perform the method, are also provided.

### CROSS-REFERENCE TO RELATED APPLICATIONS

The present application is a national phase filing under 35 U.S.C. § 371 of International Application No. PCT/EP2023/056250, filed on Mar. 10, 2023, which claims the benefit of and priority to European Patent Application No. 22382237.0, filed on Mar. 11, 2022, and European Patent Application No. 22383135.5, filed on Nov. 25, 2022, all of which are incorporated herein by reference in their entireties.

### INCORPORATION BY REFERENCE

This application contains a sequence listing entitled “P6037PC00_SeqListST26_v2.xml” being submitted herein in xml format, which was amended on Apr. 23, 2025, and is 13,810 bytes in size.

### FIELD OF THE INVENTION

The present invention relates to the fields of medicine and diagnostic or determination of the risk of developing neurodegenerative diseases and particularly, to methods for the diagnosis or determination of the risk of developing Alzheimer's disease dementia.

### BACKGROUND ART

Alzheimer's disease (AD) is a neurodegenerative disease estimated to affect around 40 million people worldwide, consequently causing a substantial societal and economic burden. At present, there is no cure nor treatment capable of decelerating the progression of AD, and only four drugs are available for symptomatic treatment. Further, clinical trials evaluating therapies for AD have faced unprecedented failures of over 99.6% since 2002, despite the high number of trials aimed to find new treatments for AD. This lack of success is a result, in part, of the recruitment of mainly patients in late stages of AD, suffering from an irreversible neurodegeneration. Thus, there is a need to shift the design of clinical trials to include patients in early stages of predementia, i.e., suffering from mild cognitive impairment (MCI).

However, a new challenge arises regarding the identification of patients at risk of developing AD. MCI-diagnosed patients indeed have a higher probability to develop AD, but only about a third of them evolve to AD. In those lines, current diagnostic methods, such as PET-amyloid imaging, PET-FDG, lumbar puncture, magnetic resonance imaging or behavioral testing methods (e.g., Clinical dementia Rating or Mini Mental State Exam), have improved recruitment efficiency, but still fail in optimally determining the risk of patients to develop AD. For instance, about a third of MCI-diagnosed patients, which are recruited due to a positive PET test, return to cognitively normal state, or develop other types of dementia. Furthermore, most of said methods are highly invasive and incur great financial costs to patients and health systems. Thus, it appears obvious that available diagnostic methods do not meet the accuracy required for clinical trials to success in evaluating potential AD treatments, nor other desirable characteristics such as non-invasiveness.

Consequently, optimal stratification of patients at high risk of developing AD during clinical trial recruitment remains a major challenge. Hence, there is a strong unmet need to develop a precise, reliable, yet feasible and cost-effective screening tool, which can identify subjects at high risk of developing AD to allow accurate recruitment and, ultimately, therapeutic development for AD. Additionally, said desirable screening approach would also allow for the differentiation of AD patients from patients suffering from other dementias, and achieve an accurate diagnostic that eases the selection of the most adequate option of treatment, when available.

Alzheimer's disease pathophysiology has been associated with alterations in mitochondrial functionality and mitochondrial DNA (mtDNA), such as inherited and somatic mutations, usually located in mtDNA regulatory elements. Consistently, there is substantial evidence of oxidative phosphorylation (OXPHOS) defects in AD, in which mtDNA-encoded polypeptides play a significant role. In those lines, AD diagnostic methods have been described based on the identification of mutations in mitochondrial DNA through restriction fragment length polymorphism (RFLP) technique or other related techniques.

WO2015/144964 A2 discloses the use of mitochondrial methylation patterns to diagnose or determine the risk to develop a neurodegenerative disease such as AD and Parkinson's disease, based on the analysis of brain samples from subjects after death. The methylation patterns disclosed in WO2015/144964 A2 result from very early stages of research. Therefore, there remains a need for a feasible, effective, and non-invasive methodology allowing for the determination of said risk at early stages of patients' dementia or even at healthy stages of subjects. Data disclosed in WO2015/144964 A2 is also discussed in Blanch et al. (2016), which attains the same conclusions, and emphasizes that methylated mtDNA represents only a small part of the total mtDNA.

The mtDNA methylation patterns in AD patients have been further studied by Stoccoro et al. (2017), which discloses a reduction in D-loop methylation levels in peripheral blood of late-onset AD patients, compared to controls. These are surprisingly differing results in comparison to earlier studies. Finally, the abstract Stoccoro et al. (2022) disclosed that patients diagnosed with MCI show higher methylation levels in the D-loop region than controls and AD patients.

Although there is evidence suggesting that mtDNA methylation, could be informative of the Alzheimer's dementia stage of a subject, its role and pattern in AD, along with its significance, is far from reaching a sharp and clear definition. Thus, its role and usefulness to determine the risk of developing Alzheimer's diseases, or to diagnose such disease at early stages remains unknown. Thus, there is a strong need to develop an effective and non-invasive method which allows for the determination of the risk to develop AD, and ultimately classify subjects correctly to ensure improved recruitment efficiency in clinical trials and adequate treatments.

### SUMMARY OF THE INVENTION

One problem to be solved by the present invention is to provide a method to diagnose or determine the risk of developing Alzheimer's disease dementia (herein referred as ADD) in a subject.

The present invention discloses a method capable of calculating or determining a score to quantify the risk of a subject to develop ADD, and consequently classify subjects according to said risk. This method comprises the execution of a classification model capable of processing more than one dataset which include biomarker screening data (i.e., mitochondrial methylation data) and other relevant clinical data (e.g., MMSE and SOB). Said biomarker screening data is obtained from blood samples, therefore allowing for a fast, non-invasive and effective methodology for the determination of such risk.

The use of mitochondrial markers to diagnose AD had already been disclosed in WO2015/144964 A2. However, Examples in WO2015/144964 A2 disclose a mitochondrial methylation pattern obtained through the analysis of a small number of brain samples (obtained after death) of subjects known to suffer from AD (N=16), along with controls (N=8). These examples analyze the methylation of a total of 89 sites which had been identified as differentially methylated by statistical methods. These sites include CpG, CHG and CHH in the D-loop region, and CpG and CHG sites in the ND1 gene.

Surprisingly, the inventors have found that the methylation sites which contribute the most to the determination of the risk of developing ADD are not disclosed in the prior art. As shown in the present invention, several new methylation sites show highly significant methylation patterns for determining such risk, mostly corresponding to CHH sites in the ND1 gene. Additionally, the inventors of the present invention have developed a set of exceptionally efficient primers for the detection of methylation in mtDNA extracted from blood samples (see EXAMPLE 1), beyond the commonly designed primers with a main focus on CpG sites. Further, examples of the present invention gather information from blood samples instead of brain samples and include a larger number of samples to develop the method disclosed herein.

Stoccoro et al. (2017 & 2022) disclose that patients diagnosed with MCI show higher levels of mitochondrial methylation in the D-loop region, whereas in AD patients said methylation levels are decreased. The abstract from Stoccoro et al. (2022) does not make distinctions between subjects at early stages of dementia (MCI), and does not disclose any information regarding how said patterns could be useful in the classification of subjects according to their risk of developing ADD. Further, again, Stoccoro et al. (2017 & 2022) do not refer either to any sites of the ND1 gene, and only to a small portion of the D-loop region disclosed herein.

Working examples herein provide detailed experimental data demonstrating an efficient processing of blood samples for the detection and calculation of mtDNA methylation. Furthermore, the information regarding said mitochondrial methylation is combined with other relevant clinical data, and altogether processed by a classification model shown to have very high performance. As a result, the method provided herein, determines a score corresponding to the risk of developing ADD, and sharply classifies subjects accordingly.

EXAMPLE 1 shows the method for detecting mtDNA methylation which comprises collecting blood samples, extracting and treating DNA (bisulfite treatment), and preparing the amplicon library to detect, quantify and normalize methylation in mtDNA sites of interest. The use of degenerated primers resulted in an extraordinarily high sensibility in the detection of mtDNA methylation, for both regions (i.e., D-loop region and ND1 gene) and in all three contexts (i.e., CpG, CHG, CHH), to the point where these results exceed any possible expectation. Further, the comparison of methylation levels between groups in terms of different contexts and regions resulted in a high number of significant differentially methylated comparisons.

EXAMPLE 2 shows the development of a classification model which considers not only data on the methylation sites of interest, but also other relevant clinical data (e.g., MMSE, SOB). The model has assigned a specific weight to each variable by statistic methods according to the inputted training data. The model is then able to calculate a score corresponding to the risk of developing ADD, with an outstanding high performance as shown by an overall accuracy score of 0.76 and a Kappa value of 0.63. Therefore, this classification model can calculate the risk of developing ADD of any subject by rapidly processing their individual information (i.e., mitochondrial methylation and clinical data) with a remarkably good performance.

It is worth noting that the model disclosed in EXAMPLE 2 did not include any clinical variable which may be obtained through invasive or highly costly techniques such as PET. In the current clinical practice, PET used to detect amyloid plaques is indeed considered a highly informative diagnostic technique for AD. However, the classification model developed herein was clearly capable of predicting the risk of developing ADD with very high-performance indicators, despite not using such information.

Further, EXAMPLE 3 shows the development of a classification model which considers data on the methylation sites of interest and other relevant clinical data as shown in EXAMPLE 2. In this case, clinical data included the above-mentioned variable PET used to detect amyloid plaques (positive or negative). This classification model was performed for those subjects who already had a PET performed, in order to take advantage of this additional information regarding the amyloid PET test (positive or negative). The model is capable of calculating a score corresponding to the risk of developing ADD, and classify patients accordingly again with an outstanding high performance as shown by an overall accuracy score of 0.89 and a Kappa value of 0.84.

EXAMPLE 4 shows the development of a classification model which considers data on the methylation sites of interest and relevant clinical data regarding the above-mentioned variable PET used to detect amyloid plaques (positive or negative). The model is capable of calculating a score corresponding to the risk of developing ADD and classify patients accordingly again with an outstanding high performance as shown by an overall accuracy score of 0.756 and a Kappa value of 0.63. Therefore, this classification model exemplifies the possibility of developing a high-performing classification model taking into consideration a low number of clinical variables, however highly contributing to determine a reliable score.

Accordingly, a first aspect of the invention relates to a method for determining the risk of developing Alzheimer's disease dementia in a subject, comprising applying a classification model to a methylation pattern of the D-loop region, and/or of the ND1 gene of the mitochondrial DNA from a sample from the subject, wherein the classification model assigns the subject a Dementia Stage Class (DSC) selected from progression to ADD or non-progression to ADD.

A second aspect of the invention relates to a method for identifying a subject suitable for treatment with a specific AD-therapy, the method comprising applying a classification model to a methylation pattern of the D-loop region, and/or of the ND1 gene of the mitochondrial DNA from a sample from the subject, wherein the classification model assigns the subject a Dementia Stage Class selected from progression to ADD or non-progression to ADD, and wherein a Dementia Stage Class consisting of progression to ADD indicates that a specific AD-therapy can be administered to the subject.

A third aspect of the invention relates to a method for treating a subject, particularly a subject diagnosed with MCI or CDR 0.5, administering a specific AD-therapy or a treatment for other dementias to the subject, wherein, prior to the administration, the subject is assigned with a DSC, determined by applying a classification model to a methylation pattern of the D-loop region, and/or of the ND1 gene of the mitochondrial DNA from a sample from the subject, wherein the DSC is selected from progression to ADD or non-progression to ADD.

A fourth aspect relates to a method for providing a personalized therapy to a subject at high risk of developing ADD comprising the steps described herein.

A fifth aspect relates to a classification model for determining the risk of developing ADD in a subject, wherein the classification model identifies a subject as pertaining to a class from the group consisting of progression to ADD and non-progression to ADD, using as data on methylation patterns obtained from a sample from the subject and data on clinical variables of the subject, and wherein being identified as ADD progression indicates that the subject is at risk of developing to ADD.

Another aspect relates to a computer-implemented method applicable to the methods described herein e.g., for obtaining a risk score of developing ADD, the method comprising the following steps:
(a) providing or receiving as input data:
   (1) the methylation pattern in the D-loop region, and/or in the ND1 gene of the mitochondrial DNA of a subject, and optionally,
   (2) at least one clinical variable of the subject, as described herein, and
(b) combining and weighting said methylation pattern(s) and clinical variable(s) using a classification model to obtain the risk score.

In some examples, input data can be received from which a computer or other data processing system can derive the methylation pattern in the D-loop region and/or the methylation pattern in the ND1 gene of the mitochondrial DNA of a subject. And the computer-implemented method can then further comprise receiving at least one clinical variable of the subject as described herein, and combining and weighting the methylation pattern(s) and clinical variable(s) using a classification model to obtain a risk score.

An aspect of the invention relates to an oligonucleotide with a length between 15 and 100 nucleotides, comprising a nucleic acid sequence selected from the group consisting of SEQ ID NO: 1, SEQ ID NO: 2, SEQ ID NO: 3, and SEQ ID NO: 4.

An aspect of the invention relates to the use of an oligonucleotide with a length between 15 and 100 nucleotides and comprising a nucleic acid sequence selected from the group consisting of SEQ ID NO: 1, SEQ ID NO: 2, SEQ ID NO: 3, and SEQ ID NO: 4 for the determination of a methylation pattern of mitochondrial DNA.

In an aspect, the invention relates to a kit comprising at least one oligonucleotide capable of specifically hybridizing with a mitochondrial DNA sequence comprising the D-loop region or the ND1 gene.

In an aspect, the invention relates to the use of the kits as defined above for the determination of a methylation pattern of mitochondrial DNA. In another aspect, the invention relates to the use of the kits as defined above, for the determination of a methylation pattern of mitochondrial DNA to determine the risk of developing Alzheimer's disease dementia in a subject. In another aspect, the invention relates to the use of the kits as defined above, following the methods as described herein.

Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. Furthermore, the present invention covers all possible combinations of particular and preferred embodiments described herein. The following examples and drawings are provided herein for illustrative purposes, and without intending to be limiting to the present invention.

### DESCRIPTION OF THE DRAWINGS

FIG. 1 shows the methylation detection of the degenerated (Deg) and Non-degenerated primers (NoDeg) primers in the detection of mtDNA methylation in the D-loop region for CpG context. FIG. 1 shows five subjects (e.g., FIS004) as examples with triplicated samples as shown in the horizontal axis.

FIG. 2 shows the methylation detection of the degenerated (Deg) and Non-degenerated primers (NoDeg) primers in the detection of mtDNA methylation in the D-loop region for CHG context. FIG. 2 shows five subjects (e.g., FIS004) as examples with triplicated samples as shown in the horizontal axis.

FIG. 3 shows the methylation detection of the degenerated (Deg) and Non-degenerated primers (NoDeg) primers in the detection of mtDNA methylation in the D-loop region for CHH context. FIG. 3 shows five subjects (e.g., FIS004) as examples with triplicated samples as shown in the horizontal axis.

FIG. 4 shows the methylation detection of the degenerated (Deg) and Non-degenerated primers (NoDeg) primers in the detection of mtDNA methylation in the ND1 gene for CpG context. FIG. 4 shows five subjects (e.g., FIS004) as examples with triplicated samples as shown in the horizontal axis.

FIG. 5 shows the methylation detection of the degenerated (Deg) and Non-degenerated primers (NoDeg) primers in the detection of mtDNA methylation in the ND1 gene for CHG context. FIG. 5 shows five subjects (e.g., FIS004) as examples with triplicated samples as shown in the horizontal axis.

FIG. 6 shows the methylation detection of the degenerated (Deg) and Non-degenerated primers (NoDeg) primers in the detection of mtDNA methylation in the ND1 gene for CHH context. FIG. 6 shows five subjects (e.g., FIS004) as examples with triplicated samples as shown in the horizontal axis.

FIG. 7 shows the bar diagrams of Dementia Stage Classification, Sex, and beta-amyloid PET variables. “DS” refers to Dementia Stage, “N” refers to “number”, “C” refers to “controls”, “NP” refers to “non-progressed”; “P” refers to “progressed”, “F” refers to “female”, “M” refers to “male”, “S” refers to “Sex”.

FIG. 8 shows the violin plots for variables Age, MMSE and SOB. “DS” refers to Dementia Stage, “A” refers to “age”, “C” refers to “controls”, “NP” refers to “non-progressed”; “P” refers to “progressed”.

FIG. 9 shows Accuracy and Kappa metrics of the supervised learning models of EXAMPLE 2.

FIG. 10 shows the ROC curve when comparing groups Progressed vs. Non-Progressed using the model built using Random Forest method in EXAMPLE 2.

FIG. 11 shows the bar diagram of PET for beta-amyloid. “N” refers to “number, “Neg” refers to negative, “Pos” refers to “positive”.

FIG. 12 shows Accuracy and Kappa metrics of the supervised learning models used in EXAMPLE 3.

FIG. 13 shows the ROC curve when comparing groups Progressed vs. Non-Progressed using the model built using Random Forest method of EXAMPLE 3.

FIG. 14 shows Accuracy and Kappa metrics of the supervised learning models used in EXAMPLE 4.

FIG. 15 shows the ROC curve when comparing groups Progressed vs. Non-Progressed using the model built using Random Forest method of EXAMPLE 4.

### DETAILED DESCRIPTION OF THE INVENTION

For the avoidance of doubt, the methods provided herein do not involve diagnosis practiced on the human or animal body. The methods of the invention are particularly conducted on a sample that has previously been extracted from the subject. The kits provided herein can include means for extracting the sample from the subject.

### Definitions

Diagnosis: The term “diagnosis” refers to both the process of trying to determine and/or identify a possible disease in a subject, that is to say the diagnostic procedure, as well as the opinion reached through this process, that is to say, the diagnostic opinion. As such, it can also be seen as an attempt to classify the status of an individual in separate and distinct categories that allow medical decisions about treatment and prognosis to taken. As will be understood by the person skilled in the art, such diagnosis may not be correct for 100% of the subjects to be diagnosed with, although it is preferred that it is. However, the term requires that a statistically significant portion of subjects can be identified as suffering from Alzheimer's disease in the context of the invention, or a predisposition thereto. The person skilled in the art may determine whether a part is statistically significant using different well known statistical evaluation tools, for example, by determining confidence intervals, determining the value of p, Student's t-test, the Mann-Whitney test, etc. Particular confidence intervals are at least 50%, at least 60%, at least 70%, at least 80%, at least 90% or at least 95%. P values are particularly 0.05, 0.025, 0.001 or lower.

Risk of developing Alzheimer's disease dementia: The term “risk of developing Alzheimer's disease dementia (ADD)” is herein used indistinctly with “risk of progressing to Alzheimer's disease dementia” and refers to the predisposition, susceptibility, or propensity of a subject to develop ADD. The risk of developing ADD generally implies that there is a high or low risk or higher or lower risk. Like that, a subject has a high risk of developing ADD has a likelihood of developing this dementia of at least 50%, or at least 60%, or at least 70%, or at least 80%, or at least 90, or at least 95%, or at least 97%, or at least 98%, or at least 99%, or at least 100%. Similarly, a subject at a low risk of developing ADD, is a subject having at least one chance of developing the dementia at most 1%, or at most 2%, or at most 3%, or at most 5%, or at most 10%, or at most 20%, or at most 30%, or at most 40%, or at most 49%.

Therefore “determining the risk of developing Alzheimer's disease dementia” refers to the probability of progressing to Alzheimer's disease dementia, comprising all possible grades of dementias within the disease.

The term “progressing to ADD”, or “ADD progressed”, or “ADD progression”; or any other similar expression is used indistinctly with “developing ADD”, or “ADD developed”, or “ADD development” or “development of ADD”.

Alzheimer's disease: The term “Alzheimer's disease” or “senile dementia” or AD refers to a mental impairment associated with a specific degenerative brain disease characterized by the appearance of senile plaques, neuritic tangles and progressive neuronal loss that is clinically manifested in progressive deficiencies of memory, confusion, behavioral problems, inability to care for oneself, gradual physical deterioration and, ultimately, death. Alzheimer's disease can be classified in the following stages according to Braak staging:
Stages I-II: the brain area is affected by the presence of neurofibrillary tangles corresponding to the transentorhinal region of the brain.
Stages III-IV: the affected brain area also extends to areas of the limbic region such as the hippocampus.
Stages V-VI: the affected brain area also involves the neocortical region.

This classification by neuropathological stages correlates with the clinical evolution of the existing disease and there is a parallel between the decline in memory with the neurofibrillary changes and the formation of neuritic plaques in the entorhinal cortex and the hippocampus (stages I to IV). Also, the isocortical presence of these changes (stages V and VI) correlates with clinically severe alterations. The transentorhinal state (I-II) corresponds to clinically silent periods of disease. The limbic state (III-IV) corresponds to a clinically incipient AD. The neocortical state corresponds to a fully developed AD.

Alzheimer's disease dementia: The term “Alzheimer's disease dementia” or “ADD” refers to the set of symptoms that includes deficiencies in memory and difficulties with thinking, problem-solving or language which develop as a result of the degenerative brain damage and progressive neuronal lost characteristic of Alzheimer's disease.

In clinical terms, those subjects at risk of developing/progressing to Alzheimer's disease in the future have to be referred to as being at risk of developing/progressing to Alzheimer's disease dementia (ADD), as this is the set of symptoms which might appear or subjects at risk might suffer from. Therefore, the present invention is directed to methods for determining the risk of developing/progressing to ADD, methods for identifying subjects at risk of developing/progressing to ADD, and other methods relating to the risk of developing/progressing to ADD.

Subject: The terms “subject”, “patient”, “individual”, and variants thereof are used interchangeably herein and refer to any mammalian subject, particularly a human subject. The term does not denote a particular age or sex.

Sample comprising mitochondrial DNA: The expression “sample comprising mitochondrial DNA” as used herein refers to any sample that can be obtained from a subject in which there is genetic material from the mitochondria suitable for detecting the methylation pattern.

Mitochondrial DNA: The term “mitochondrial DNA” or “mtDNA” as used herein, refers to the genetic material located in the mitochondria of living organisms. It is a closed, circular double-stranded molecule. In humans it consists of 16,569 base pairs, containing a small number of genes, distributed between the H chain and L chain. Mitochondrial DNA encodes 37 genes: two ribosomal RNA, 22 transfer RNA and 13 proteins that participate in oxidative phosphorylation.

Methylation pattern and methylation status: The term “methylation pattern” as used herein refers but is not limited to the presence or absence of methylation of one or more nucleotides, particularly the methylation in cytosines. In this way, said one or more nucleotides are comprised in a single nucleic acid molecule. Said one or more nucleotides are capable of being methylated or not. The term “methylation status” can also be used when only considered a single nucleotide. A methylation pattern can be quantified; in the case it is considered more than one nucleic acid molecule.

D-loop region: The term “D-loop region” as used herein, refers to a region of non-coding mtDNA, which acts as a promoter for both the heavy and the light strains of the mtDNA, and contains essential transcription and replication elements. The D-loop region contains approximately 1120 base pairs, visible under electron microscopy, which is generated during H chain replication for the synthesis of a short segment of the heavy strand, 7S DNA. The human D-loop region sequence is deposited in the GenBank database under the accession number NC_012920.1.

ND1 gene: The term “ND1 gene” or “NADH dehydrogenase 1” or “ND1mt”, as used herein, refers to the gene localized in the mitochondrial genome that encodes the protein NADH dehydrogenase 1 or ND1. The human ND1 gene sequence is deposited in the GenBank database under the accession number NC_012920.1. The ND1 protein is part of the enzyme complex called complex I which is active in the mitochondria and is involved in the process of oxidative phosphorylation. In some embodiments, the term “ND1 gene” may refer to the gene above further comprising comprise approximately 50 additional base pairs in one or the two extremes of the sequence.

CpG site: The term “CpG site” as used herein, to distinguish this single-stranded linear sequence from the CG base-pairing of cytosine and guanine for double-stranded sequences. “CpG” is an abbreviation for “C-phosphate-G”, i.e., cytosine and guanine separated by only a phosphate; phosphate binds together any two nucleosides in the DNA. The term “CpG” is used to distinguish this linear sequence of CG bases pairing of guanine and cytosine. Cytosine in the CpG dinucleotides may be methylated to form 5-methylcytosine.

CHG site: The term “CHG site” as used herein, refers to DNA regions, particularly mitochondrial DNA regions, where a cytosine nucleotide and a guanine nucleotide are separated by a variable nucleotide (H) which can be adenine, cytosine, or thymine. The cytosine of the CHG site can be methylated to form 5-methylcytosine.

CHH site: The term “CHH site” as used herein, refers to DNA regions, particularly regions of mitochondrial DNA, where a cytosine nucleotide is followed by a first and a second variable nucleotide (H) which can be adenine, cytosine, or thymine. The cytosine of the CHG site can be methylated to form 5-methylcytosine.

Determination of the methylation pattern in a CpG site: The term “determination of the methylation pattern in a CpG site” as used herein, refers to the determination of the methylation status of a particular CpG site. The determination of the methylation pattern of a CpG site can be performed by multiple processes known to the person skilled in the art.

Determination of the methylation pattern in a CHG site: The term “determination of the methylation pattern in a CHG site” as used herein, refers to the determination of the methylation status of a particular CHG site. The determination of the methylation pattern of a CHG site can be performed by multiple processes known to the person skilled in the art.

Determination of a methylation pattern in a CHH site: The term “determination of a methylation pattern in a CHH site”, as used herein, refers to determining the methylation status of a particular CHH site. The determination of the methylation pattern of a CHG site you can be performed by multiple processes known to the person skilled in the art.

To determine the methylation pattern in mitochondrial DNA, samples can be chemically treated so that all cytosine unmethylated bases are modified at uracil bases, or another base which differs from cytosine in terms of base pairing behavior, while the bases of 5 methylcytosine remain unchanged. The term “modify” as used herein means the conversion of an unmethylated cytosine to another nucleotide that will distinguish the unmethylated cytosine from the methylated cytosine. The conversion of unmethylated cytosine bases, but not methylated, in the sample containing mitochondrial DNA is carried out with a conversion agent. The term “conversion agent” or “conversion reagent” as used herein, refers to a reagent capable of converting an unmethylated cytosine to uracil or another base that is differentially detectable to cytosine in terms of hybridization properties. The conversion agent is particularly a bisulfate such as bisulfites or hydrogen sulfite. However, other agents that similarly modify unmethylated cytosine, but not methylated cytosine can also be used in this method of the invention, such as hydrogen sulfite. The reaction is performed according to standard procedures (Frommer et al., 1992, Proc. Natl. Acad. Sci. USA 89:1827-1831; Olek, 1996, Nucleic Acids Res. 24:5064-6; EP 1394172). It is also possible to carry out the conversion enzymatically, e.g., using cytidine deaminases specific methylation.

Reference sample: The term “reference sample” refers to a sample containing mitochondrial DNA obtained from a subject not suffering from AD. In particular, said term refers to a small number of 5-methylcytosines in one or more CpG sites in the D-loop region shown in Table 1, in one or more CpG sites of the ND1 gene shown in Table 2, in one or more sites CHG sites in the D-loop region shown in Table 3, in one or more CHG sites in the ND1 gene shown in Table 4, one or more CHH sites in the D-loop region shown in Table 5 and/or one or more CHH sites in the ND1 gene shown in Table 6 in a sequence of mitochondrial DNA as compared to the relative amount of 5-methylcytosines present in said one or more CpG sites, one or more CHG sites and/or one or more CHH sites in a reference sample.

Treatment of Alzheimer's disease: The term “treatment of AD”, as used herein, refers to treatment for the disease or any related symptoms. Such treatments can include medications, epigenetic treatments, or any cognitive stimulating treatment. Some cognitive stimulating treatments are digital cognitive treatments (i.e., using digital devices). This term can include any treatment known in the art for AD or future developments. Treatments of AD can include, but are not limited to antioxidants, anti-inflammatory drugs, Ginkgo biloba, vitamin or dietary supplements, and antibodies against beta-amyloid like Aducanumab or other similar.

Further, in the present invention, subjects can be classified as being at risk of developing ADD in the future. Some treatments for AD described above can be useful for the treatment of these patients to delay or prevent the onset of symptoms of AD. Other treatments can specifically be useful for the delay or prevention of the onset of symptoms of AD. The treatment of AD administered to subjects which have not yet developed ADD but are at risk of developing ADD (i.e., as determined by the methods disclosed herein) may be referred to as “preventive treatment of AD”, as used herein. Therefore, as used herein, the term “treatment of AD” may refer also to “preventive treatment of AD”.

Preventive treatment of AD: The term “preventive treatment”, as used herein, refers to the prevention or conjunction of prophylactic measures to prevent or delay the onset of symptoms, as well as reducing or alleviating clinical symptoms thereof. In particular, the term refers to the prevention or set of measures to prevent the occurrence, to delay or to relieve the clinical symptoms associated with Alzheimer's disease. Desired clinical outcomes associated with the administration of the treatment to a subject include but are not limited to, stabilization of the pathological stage of the disease, delay in the progression of the disease and improvement in the physiological state of the subject. Suitable preventive treatments aimed at preventing or delaying the onset of the symptoms of Alzheimer's disease include but are not limited to cholinesterase inhibitors such as donepezil hydrochloride (Arecept), rivastigmine (Exelon) and galantamine (Reminyl) or antagonists N-methyl-D-aspartate (NMDA).

Dementia Stage Classification (DSC): The term “Dementia Stage Classification”, as used herein, refers to the classification of subjects based on the prediction of the evolution of their dementia. This classification results from the risk score of progressing to ADD as determined by the classification model disclosed herein. The classification model according to examples of the present invention classifies subjects in those who are expected to or diagnosed to develop Alzheimer's disease dementia (progression to ADD) and those subjects who are expected to or diagnosed not to develop Alzheimer's disease dementia (non-progression to ADD). Subjects who will not develop Alzheimer's disease dementia may remit their dementia, establish with a mild stage of dementia, or develop other types of dementia. Therefore “Dementia Stage Classification” includes two classes: progression to ADD and non-progression to ADD.

Further, Dementia Stage Classification (DSC) is also mentioned in the development of the classification model, particularly during the training process of the development. In this case, the Dementia Stage Classification (DSC) can include a third class: control. This is because the training dataset used for the development of the classification model used subjects which actually developed to ADD, subjects that did not progress to ADD and controls, which did not suffer from any mild dementia nor progressed to ADD. Therefore, the classes included in the Dementia Stage Classification (DSC) may be also referred as ADD progressed (i.e., progression to ADD) and ADD non-progressed (i.e., non-progression to ADD).

Specifically hybridizes: The expression “specifically hybridizes” or “capable of hybridizing in a specific form”, as used herein, refers to the ability of an oligonucleotide or of a polynucleotide of specifically recognizing a specific sequence of interest, e.g., D-loop region or ND1 gene. The sequence of interest may refer to the reference sequence or the sequence resulting from certain modification treatment, e.g., bisulfite treatment wherein unmethylated cytosines are modified to uracil. As used herein the term “hybridization”, refers the process of combining two nucleic acid molecules or single-stranded molecules with a high degree of similarity resulting in a simple double-stranded molecule by specific pairing between complementary bases. Normally hybridization occurs under very stringent conditions or moderately stringent conditions.

Oligonucleotide: The term “oligonucleotide” as used herein, is used indistinctly with “primer” and “nucleic acid sequence” and refers to a DNA molecule or short RNA, with up to 100 bases in length.

Oligonucleotides of the invention are particularly DNA molecules at least 2, at least 3, at least 4, at least 5, at least 6, at least 7, at least 8, at least 9, at least 10, at least 15, at least 20, at least 30, at least 40, at least 50, at least 60, at least 70, at least 80, at least 90 or 100 bases of length.

Score: The term “score” refers to one or more values, particularly a single value, that can be used as a component in a classification model for the determination of the risk to develop a disease for a subject. Such a single value can be calculated or determined (i.e., estimated) by combining the values of descriptive features processed by an interpretation function or algorithm. In examples of the present invention, the score relating to the risk to develop ADD (also referred to “risk score”) refers to the probability to the risk of developing ADD. Risk scores can be scores of 0.0-1, with 0 indicating lowest risk of developing the disease and 1 indicating highest risk of developing the disease. Risk scores can be classified in groups, i.e., classes, such as non, low, intermediate, and high.

Classifying a subject according to the risk of developing Alzheimer's disease dementia: The term “classifying a subject according to the risk of developing Alzheimer's disease dementia” means assigning a diagnostic subcategory herein referred to dementia stage classification, which can include at least two categories: progression to ADD (referring to subjects at risk of progressing to ADD) and non-progression to ADD (referring to subjects not at risk of progressing to ADD).

Computer-implemented methods: The term “computer-implemented method” refers to methods in which all or some steps of a method are carried out by a computer, another programmable apparatus, or a network of computers.

Supervised machine learning methods: The term “supervised learning methods” refers to methods which use a training set to generate a desired output to develop a model. This training dataset includes inputs and correct outputs, which allow the model to learn over time. Its accuracy is measured through a loss function, and its model can be adjusted until a generalization error has been sufficiently minimized.

Non-supervised machine learning methods: The term “non-supervised learning methods”, also referred as “unsupervised learning methods” refer to methods which use machine learning algorithms to analyze unlabeled datasets. These methods recognize similarities and differences in information to detect data groups or patterns. These methods are commonly used for clustering, association, and dimensionality reduction of datasets.

Deep learning methods: The term “Deep learning methods” refers to a subfield of machine learning that uses neural networks models with multiple layers to automatically extract and learn features and patterns from raw data. A layer in a neural network is a set of interconnected artificial neurons (aka nodes). These neurons receive input data and apply specific types of computational tasks to it. Usually, these tasks involve weighted sums and mathematical activation functions. The output of a layer is passed to the next layer and so on. This process allows the network to gradually learn more complex features, attributes and patterns in the data. There are different types of layers in a neural network. Some examples are fully connected layers, convolutional layers, pooling layers, and recurrent layers.”

Artificial intelligence methods: The term “artificial intelligence methods” refers to methods using artificial intelligence, defined as the capacity of a computer or a computer-controlled robot to emulate human's capabilities to respond to certain stimuli.

Classification model: The term “classification model” is herein indistinctly referred to as “classifying model”. The classification model is a model developed using for e.g., a type of supervised learning method capable of accurately assigning test data/new observations into specific categories based on training data. The model is trained to learn from the given training dataset and is consequently capable to classify new datasets into certain score/number or class/group. Examples of classification algorithms are Linear Discriminant Analysis (LDA), Classification and Regression Trees (CART), k-Nearest Neighbors (kNN), Naive Bayes (NB), Support Vector Machines (SVM) with a linear kernel, Random Forest (RF), and Neural Network (NNET).

Converting: The term “converting” means subjecting the one or more descriptive features to an interpretation function or algorithm for a predictive model of disease, particularly Alzheimer's disease. In some embodiments, the interpretation function can also be produced by a plurality of predictive models. In one embodiment, the predictive model includes a regression model and a Bayesian classifier or score. In one embodiment, an interpretation function comprises one or more terms associated with one or more biomarkers or sets of biomarkers. In one embodiment, an interpretation function comprises one or more terms associated with the presence or absence or spatial distribution of the specific cell types disclosed herein. In one embodiment, an interpretation function comprises one or more terms associated with the presence, absence, quantity, intensity, or spatial distribution of the morphological features of a cell in a cell sample. In one embodiment, an interpretation function comprises one or more terms associated with the presence, absence, quantity, intensity, or spatial distribution of descriptive features of a cell in a cell sample.

### Methods and Classification Model

One aspect of the invention relates to a method for determining the risk of developing Alzheimer's disease dementia in a subject, comprising applying a classification model to a methylation pattern of the D-loop region, and/or of the ND1 gene of the mitochondrial DNA from a sample from the subject, wherein the classification model assigns the subject a Dementia Stage Class (DSC) selected from progression to ADD or non-progression to ADD. A computer-implemented method for determining the risk of developing Alzheimer's disease dementia in a subject can comprise receiving a methylation pattern of the D-loop region and/or of a ND1 gene of the mitochondrial DNA from a sample from the subject, and a classification model assigning a DSC to the subject.

In some embodiments, the method comprises applying the classification model to at least one clinical variable. This method can alternatively be formulated as a method for predicting the development of ADD in a subject or a method for determining/identifying/assigning a Dementia Stage Class to a subject; or as a method for differentiating between a high risk and a low risk of a subject to develop ADD.

Alternatively, the invention relates to a method, particularly a computer-implemented method, of determining the risk of developing ADD in a subject, comprising: (a) determining a methylation pattern in (a) the D-loop region, and/or (b) the ND1 gene of mitochondrial DNA from a sample obtained from the subject; and (b) determining a risk score indicative of the risk of developing ADD, wherein the risk score is calculated using a classification model configured to combine the methylation pattern of one or more sites determined in step (a) with at least one clinical variable of the subject, as described herein. This method can alternatively be formulated as a method for identifying a human subject at risk of developing ADD.

The methods described herein (based on determining the risk of developing Alzheimer's disease dementia in a subject) are useful for the diagnosis of ADD in a subject. The predictive methods described herein can be used clinically to make treatment decisions by choosing the most appropriate treatment modalities for any particular patient. The methods are also useful for classifying a subject according to the risk of developing ADD in order e.g., to enter in a clinical trial or to receive adequate treatment in an early stage, i.e., prior to the development of severe dementia, corresponding to the development of ADD. Thus, the application of the methods disclosed herein can improve clinical outcomes by matching patients to therapies and also improve the accuracy in the selection of patients required for clinical trials to success in evaluating potential AD treatments. The methods described herein are also useful for monitoring the progression to ADD in a subject over time or for monitoring the risk of developing ADD in a subject over time. Such methods can be also referred to methods to determine the prognosis.

In that sense, another aspect relates to a method, particularly a computer-implemented method, for identifying a subject suitable for treatment of AD, the method comprising applying a classification model to a methylation pattern of the D-loop region, and/or of the ND1 gene of the mitochondrial DNA from a sample from the subject, wherein the classification model assigns the subject a Dementia Stage Class selected from progression to ADD or non-progression to ADD, and wherein a Dementia Stage Class consisting of progression to ADD indicates that a treatment for AD can be administered to the subject. If the subject is assigned as non-progression to ADD, the subject may not be subjected to any treatment but undergo clinical follow-up. Alternatively, the subject may be treated for other dementias if required. This aspect can alternatively be formulated as relating to a method of classifying a subject according to the risk of developing ADD comprising following the steps described herein; a method for selecting a subject at risk of developing ADD to receive therapy, comprising the steps described herein; a method of selecting a suitable therapy to treat a subject at high risk of developing ADD comprising the steps described herein.

Alternatively, the invention relates to a method, particularly a computer-implemented method, for selecting a human subject for a treatment (such as a prophylactic treatment) for Alzheimer's disease, comprising: (a) determining a methylation pattern in (a) the D-loop region, and/or (b) the ND1 gene of mitochondrial DNA from a sample obtained from the subject; and (b) determining a risk score indicative of the risk of developing ADD, wherein the risk score is calculated using a classification model configured to combine the methylation pattern of each site determined in step (a) with at least one clinical variable of the subject, as described herein.

Besides, other aspects relate to a method, particularly a computer-implemented method, for treating a subject, particularly a subject diagnosed with MCI or CDR 0.5, administering a treatment for AD, wherein, prior to the administration, the subject is assigned with a DSC, determined by applying a classification model to a methylation pattern of the D-loop region, and/or of the ND1 gene of the mitochondrial DNA from a sample from the subject, wherein the DSC is selected from progression to ADD or non-progression to ADD. In another particular embodiment, the subject has AD or is at risk of developing ADD. In an embodiment, when a subject is assigned with DSC progression to ADD, the subject is suitable to be administered with a treatment for AD. In another embodiment, if the subject is assigned as non-progression to ADD, the subject may not be subjected to any treatment but undergo clinical follow-up. Alternatively, the subject may be treated for other dementias if required. This can alternatively be formulated as a method, particularly a computer-implemented method, for treating a subject comprising:
(i) assigning, prior to the administration, a subject a DCS applying a classification model to a methylation pattern of the D-loop region, and/or of the ND1 gene of the mitochondrial DNA from a sample from the subject, wherein the DSC is selected from progression to ADD or non-progression to ADD; and,
(ii) administering to the subject a treatment for AD if DSC is progression to ADD or no-treatment for ADD or clinical follow-up or administration of a therapy for other dementias if DSC is non-progression to ADD.

Alternatively, the invention relates to a method, particularly a computer-implemented method, of treating a subject having Alzheimer's disease or at risk of developing ADD, comprising: (a) determining a methylation pattern in (a) the D-loop region, and/or (b) the ND1 gene of mitochondrial DNA from a sample obtained from the subject; (b) determining a risk score indicative of the risk of developing ADD, wherein the risk score is calculated using a classification model configured to combine the methylation pattern of one or more sites determined in step (a) with at least one clinical variable of the subject, as described herein; and, (c) administering a treatment to the subject if the risk score indicates that the subject is at the risk of developing ADD.

Another aspect relates to a method, particularly a computer-implemented method, for providing a personalized therapy to a subject at high risk of developing ADD comprising the steps described herein.

Another aspect relates to a method, particularly a computer-implemented method, to monitor the progression of Alzheimer's disease in a subject or to monitor the risk of developing ADD in a subject, comprising: (a) determining a methylation pattern in (a) the D-loop region, and/or (b) the ND1 gene of mitochondrial DNA from a sample obtained from the subject; (b) determining a risk score indicative of the risk of developing ADD, wherein the risk score is calculated using a classification model configured to combine the methylation pattern of one or more sites determined in step (a) with at least one clinical variable of the subject, as described herein; and, (c) comparing the risk score determined in step (b) with the risk score obtained in an earlier stage of the disease. A risk score higher than the previous risk score is indicative of the advance of ADD, and thus, a bad prognosis. The risk score can me monitored e.g., once a year.

In some embodiments, the methods described above comprise:
a) determining in a sample of the subject comprising mitochondrial DNA, a methylation pattern in the D-loop region, and/or in the ND1 gene of the mitochondrial DNA; and
b) combining the methylation pattern data with at least one clinical variable of the subject, as described herein;
wherein said combining is performed using a classification model for determining a risk score which correlates to the risk of developing ADD in the subject.

These steps can alternatively be formulated as:
a) determining in a sample of the subject comprising mitochondrial DNA, a methylation pattern in the D-loop region, and/or in the ND1 gene of the mitochondrial DNA; and
b) determining a risk score indicative of the risk of developing ADD, wherein the risk score is calculated using a classification model configured to combine the methylation pattern of one or more sites determined in step (a) with at least one clinical variable of the subject, as described herein.

In some embodiments, the methylation pattern in the D-loop region, and/or in the ND1 gene of the mitochondrial DNA is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6.

In some embodiments, the methylation pattern is determined using at least one oligonucleotide capable of specifically hybridizing with a mitochondrial DNA sequence comprising a methylation site selected from the group consisting of (i)-(vi).

TABLE 1
List of CpG sites (i.e., positions) from
16491 and 202 in the D-loop region.

| | |
|---|---|
| CpG sites in | 16495, 16542, 16565, 33, 61, 78, 80, 91, 96, 105, 120, |
| the D-loop | 162, 170 and 186. |
| region | |

TABLE 2
List of CpG sites from 3284 and 3657 in the ND1 gene.

| | |
|---|---|
| CpG sites in | 3351, 3375, 3379, 3406, 3420, 3435, 3453, 3459, 3495, |
| the ND1 gene | 3525, 3530, 3549 and 3642. |

TABLE 3
List of CHG sites from 16491 and 202 in the D-loop region.

| | |
|---|---|
| CHG sites in | 16494, 16501, 16514, 6, 64, 98, 104, 122, 128, 141 and |
| the D-loop | 182. |
| region | |

TABLE 4
List of CHG sites from 3284 and 3657 in the ND1 gene.

| | |
|---|---|
| CHG sites in | 3374, 3455, 3494, 3524, 3529, 3589, 3641 and 3657. |
| the ND1 gene | |

TABLE 5
List of CHH sites from 16491 and 202 in the D-loop region.

| | |
|---|---|
| CHH sites in | 16498, 16507, 16508, 16511, 16520, 16527, 16528, 16536, 16537, 16538, 16540, |
| the D-loop | 16546, 16547, 16548, 16549, 16560, 16563, 4, 11, 15, 17, 18, 19, 26, 27, 29, 31, |
| region | 39, 41, 43, 44, 48, 76, 86, 110, 112, 113, 114, 132, 140, 144, 145, 147, 150, 151, |
| | 164, 166, 167, 174, 190, 194 and 198. |

TABLE 6
List of CHH sites from 3284 and 3657 in the ND1 gene.

| | |
|---|---|
| CHH sites in | 3287, 3292, 3293, 3295, 3298, 3303, 3306, 3310, 3311, 3312, 3317, 3318, 3321, |
| the ND1 gene | 3322, 3324, 3325, 3328, 3330, 3331, 3333, 3340, 3341, 3342, 3346, 3353, 3359, |
| | 3363, 3364, 3370, 3388, 3393, 3400, 3403, 3408, 3414, 3415, 3416, 3417, 3429, |
| | 3430, 3431, 3432, 3439, 3442, 3445, 3448, 3449, 3450, 3461, 3462, 3469, 3471, |
| | 3474, 3476, 3477, 3484, 3485, 3486, 3487, 3493, 3497, 3498, 3500, 3503, 3506, |
| | 3507, 3510, 3512, 3513, 3514, 3516, 3519, 3522, 3527, 3528, 3533, 3534, 3539, |
| | 3541, 3543, 3545, 3546, 3551, 3553, 3556, 3559, 3566, 3567, 3568, 3569, 3570, |
| | 3571, 3573, 3574, 3575, 3576, 3580, 3581, 3582, 3585, 3586, 3587, 3588, 3594, |
| | 3597, 3598, 3600, 3603, 3604, 3609, 3610, 3612, 3613, 3622, 3626, 3627, 3629, |
| | 3630, 3632, 3636, 3637, 3648, 3650, 3654 and 3655. |

In some embodiments, the methylation pattern is determined using at least one oligonucleotide capable of specifically hybridizing with a mitochondrial DNA sequence comprising a methylation site selected from the group consisting of (i)-(vi). Particularly the at least one of oligonucleotides has a length between 15 and 100 nucleotides and comprises a sequence selected from the group consisting of SEQ ID NO: 1, SEQ ID NO: 2, SEQ ID NO: 3 and SEQ ID NO: 4.

In an embodiment, the methods described herein comprise:
a) determining in a sample of the subject comprising mitochondrial DNA, the methylation pattern in the D-loop region, and/or in the ND1 gene of the mitochondrial DNA, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6; and
b) using a classification model for determining a risk score which correlates the methylation patterns to the risk of developing ADD in the subject.

In an embodiment, the methods described herein comprise:
a) determining in a sample of the subject comprising mitochondrial DNA, the methylation pattern in the D-loop region, and/or in the ND1 gene of the mitochondrial DNA, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6;
wherein the methylation pattern correlates to the risk of developing ADD in the subject.

In some embodiments, the methods of the invention comprise:
a) determining in a sample of the subject comprising mitochondrial DNA, the methylation pattern in the D-loop region, and/or in the ND1 gene of the mitochondrial DNA, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6; and
b) combining the methylation pattern of one or more sites determined in step (a), with at least one clinical variable of the subject, as described herein;
wherein said combining is performed using a classification model for determining a risk score which correlates to the risk of developing ADD in the subject.

Particularly, the methylation pattern is determined using at least one oligonucleotide/primer capable of specifically hybridizing with a mitochondrial DNA sequence comprising a methylation site selected from the group consisting of (i)-(vi).

### Classification Model

In another aspect, the present invention provides a classification model that is able to classify subjects into progression to ADD and non-ADD progression classes. These classes are associated to a subject at risk of developing ADD and a subject not at risk of developing ADD, respectively.

Thus, the present invention provides a classification model for determining the risk of developing ADD in a subject, wherein the classification model identifies a subject as pertaining to a class from the group consisting of progression to ADD and non-progression to ADD, using as data on methylation patterns obtained from a sample from the subject and data on clinical variables of the subject, and wherein being identified as ADD progression indicates that the subject is at risk of developing to ADD.

In some embodiments, the methods disclosed herein involve determining a risk score indicative of the risk of developing ADD, wherein the risk score is calculated or determined using a classification model configured to combine a methylation pattern with at least one clinical variable of the subject, as described herein.

In some embodiments, the methylation patterns comprise at least one methylation pattern in the D-loop region and/or in the ND1 gene of the mitochondrial DNA, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6.

In some embodiments, the classification model is obtained by methods of artificial intelligence. In a particular embodiment, the classification model is obtained by machine learning methods. In a more particular embodiment, the classification model is obtained by a supervised machine learning method. In a more particular embodiment, the supervised machine learning method is selected from the group consisting of Linear Discriminant Analysis LDA), Penalized Multinomial Regression (PMR), Classification and Regression Trees (CART), k-Nearest Neighbors (kNN), Naive Bayes (NB), Support Vector Machines (SVM) with a linear kernel, Support Vector Machines with Radial Basis Function Kernel (SVM.Radial), Random Forest (RF), and Neural Network (NNET), Logistic Regression, Artificial Neural Network (ANN), GBoost (XGB; an implementation of gradient boosted decision trees designed for speed and performance), Glmnet (a package that fits a generalized linear model via penalized maximum likelihood), cforest (implementation of the random forest and bagging ensemble algorithms utilizing conditional inference trees as base learner), Treebag (bagging, i.e., bootstrap aggregating, algorithm to improve model accuracy in regression and classification problems which building multiple models from separated subsets of train data, and constructs a final aggregated model), or a combination thereof. More particularly, the supervised machine learning method is selected from the group consisting of Linear Discriminant Analysis (LDA), Penalized Multinomial Regression (PMR), Classification and Regression Trees (CART), k-Nearest Neighbors (KNN), Naive Bayes (NB), Support Vector Machines (SVM) with a linear kernel, Support Vector Machines with Radial Basis Function Kernel (SVM.Radial), Random Forest (RF), and Neural Network (NNET). More particularly, the supervised machine learning method is Random Forest. In another embodiment, the classification model is obtained by a non-supervised machine learning method. In a particular embodiment, the non-supervised machine learning method is selected from the group consisting of K-means, K-Medoids, Fuzzy C-Means, Agglomerative Hierarchical Clustering, Gaussian Mixture Model (GMM), Neural Networks, Hidden Markov Model (HMM), Mean-Shift, DBSCAN Clustering, Apriori algorithm, Principle Component Analysis (PCA), Independent Component Analysis (ICA) Linear Discriminant Analysis (LDA), Singular Value Decomposition (SVD), Linear Semantic Analysis (LSA), t-Distributed Stochastic Neighbor Embedding (t-SNE), Nonlinear Multidimensional Scalling, Principal Curves, k-nearest neighbors (kNN), Locally Kinear Embedding and Autoencoders.

Alternatively, the classification model is obtained by a deep learning method. In a particular embodiment, the deep learning method can be supervised or non-supervised. In another embodiment, the deep learning method is selected from the group consisting of Convolutional Neural Networks (CNNs), Long Short-Term Memory Networks (LSTMs), Recurrent Neural Networks (RNNs), Generative Adversarial Networks (GANs), Radial Basis Function Networks (RBFNs), Multilayer Perceptrons (MLPs), Self-Organizing Maps (SOMs), Deep Belief Networks (DBNs), Restricted Boltzmann Machines (RBMs) and autoencoders.

In some embodiments, the classification model is trained or has been trained with a training set comprising mitochondrial methylation patterns for the methylation sites as defined herein in a plurality of samples associated with a plurality of subjects and comprising clinical variables associated to a plurality of subjects, wherein each subject is assigned a Dementia Stage Classification. In a particular embodiment, the Dementia Stage Classification is selected from the group consisting of control, ADD progressed and ADD non-progressed.

In some embodiments, subjects classified as controls are characterized by having a CDR score of 0 and a clinical follow-up longer than 10 years (i.e., more than 10 years of clinical follow-up). In some embodiments, subjects classified as ADD non-progressed have a CDR score of 0.5 and a clinical follow-up longer than 36 months without showing progression of symptoms. In some embodiments, subjects classified as ADD progressed have a CDR score of 1 after progressing from 0.5.

In some embodiments, the training dataset comprises correct outputs which correspond to the Dementia Stage Class assigned to each subject, wherein the dementia stage class are controls, progression to ADD and non-progression to ADD.

In some embodiments, data is pre-processed or has been pre-processed before the classification model is trained. This step is conducted to ensure and enhance the performance of the model training process. The data pre-processing comprises (1) creating dummy variables, (2) removing zero- and near zero-variance variables, (3) dimensionality reduction, (4) splitting the data into a training and testing data sets, (5) centering and scaling, and (6) examining and visualizing the training data set.

Particularly, (1) creating dummy variables process is conducted in order to handle categorical data. Basically, each categorical variable is transformed to a numerical variable by creating dummy variables using the called “one-hot encoding” approach (i.e., each new variable is coerced to have a value of either 0 or 1, representing the presence or absence of that attribute). The process is performed to ensure that variables are encoded to be consistent. That is, it is coded to guarantee that there are no linear dependencies between the new attributes and thus avoid the dummy variable trap.

Additionally, the data is reviewed to ensure that all categorical 1-coded variables do not show any anomalous linear combinations, if so, redundant variables are removed until eliminating the linear combinations.

(2) Removing zero- and near zero-variance variables process is conducted to remove variables showing a single unique value, and variables having a few unique numeric values that are highly unbalanced. Otherwise, these predictors might cause instability issues during the fitting process or the model crashing.

(3) Dimensionality reduction process is performed to reduce the number of variables (i.e., features or attributes) in the data set while retaining as much as relevant information as possible. In other words, the objective is to remove redundant or irrelevant features with view to improve the efficiency and effectiveness of the learning method applied to build the classification model. In this context there are two main approaches: the feature selection and the feature extraction.
(i) Feature selection techniques: The basic idea of these methods is to select a subset of variables based on some criteria, e.g., by identifying and removing correlated variables. This process is conducted with the purpose of reducing highly correlated variables. To perform this step, a correlation matrix is calculated. Usually, the correlation measure applied is the Pearson's Correlation Coefficient. Then, to detect the highly correlated variables, based on the absolute values of pairwise correlations, if two variables have a high correlation, one looks at the mean absolute correlation of each variable and removes the variable with the largest mean absolute correlation. In this context, usually, the pairwise absolute correlation cutoff can be setup by studying, for example, the linear regression between each pair of variables. However, this approach could fail to reveal additional feature correlations. For this reason, on one hand, other monotonic methods can be also considered to identify the correlation between variables. For example, the non-parametric methods Spearman's Rank Correlation and Kendall's Tau Correlation measure the association between each pair of variables that are based on the ranks of the observations rather than their actual values. They are usually applied when there are reasonable suspicious of nonlinear relationship between the attributes (variables), skewed or ordinal measures. Other type of method can be the distance correlation (aka dCor). It is performed to measure the dependence between each pair of variables in a way that is sensitive to nonlinear relationships. Most of these methods are thought to study the relationship between continuous and/or ordinal variables but also there are methods to study the strength of the relationship between a dichotomous variable and a continuous variable (e.g., the Point Biserial Correlation) or when both variables are dichotomous (Phi Coefficient). On the other hand, in order to determine an appropriate threshold for detecting the highly correlated variables, it is recommended to use an objective approach such as a cross-validation. That is, to determine the required cutoff, first, one must split the data into training and testing data sets using a random seed to ensure the reproducibility. Second, train the model using the training data set and evaluate its performance using the testing set. Third, vary the threshold for considering the variables as highly correlated and then repeat the second step for each threshold value. Fourth, select the threshold that generates the best performance on the testing set, such as the one with the highest accuracy (or lowest error rate). Fifth, after having select the cutoff, it must be applied to the entire dataset and re-train the model using all the data.

Other feature selection methods that can be applied are the Genetic Algorithms (GA), L1-regularized logistic regression, Lasso regression or hybrid methods.

(ii) Feature extraction techniques: These approaches generate new variables by combining or applying a transformation on the original features into a reduced dimension space. Some examples are the Principal Components (PCA), Multiple Factor Analysis (MFA), t-SNE or alternatively UMAP and/or Multidimensional Scaling (MDS) approaches, Partial Least Square-Discriminant Analysis (PLS-DA) or Autoencoders.

(4) Data splitting process is performed to randomly split into two main subsets: one for performing the model training (80% of the samples) and other for testing the classification model (20% of the samples). The random sampling process is driven within each class to preserve the overall class distribution of the data. A random seed is considered in order to ensure the reproducibility.

(5) Centering and scaling process is applied on the continuous features (variables) of the training data set with a view to estimate the centering and scaling factors that must be applied on both data sets to generate the normalized data sets for performing the training and testing processes of the classification model.

(6) Examining and visualizing the training data set is a process carried out after having performed the previous preprocessing tasks. It is a second exploration data analysis (EDA) performed on the training data set and guided to review and check no biases from the original data. Classical statistical descriptive methods are applied (that is univariate, bivariate and multivariate descriptive methods).

The classification models disclosed herein can be trained with data corresponding to a set of samples for which methylation data corresponding to a set of methylation sites has been obtained. For example, a training set comprises methylation pattern data from the methylation sites presented in Tables 1-6, or any combination thereof. In some embodiments, the methylation pattern data comprises data of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 methylation sites. In some embodiments, the methylation pattern data comprises data of more than 50 methylation sites. In some embodiments, the methylation pattern data comprises data of more than 100 methylation sites. In some embodiments, the methylation pattern data comprises data of more than 200 methylation sites. In some embodiments, the methylation pattern data comprises between about 10 and about 20, about 20 and about 30, about 30 and about 40, about 40 and about 50, about 50 and about 60, about 60 and about 70, about 70 and about 80, about 80 and about 90, about 90 and about 100, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, between about 240 and about 250 methylation sites selected from Tables 1-6.

In some embodiments, the training dataset comprises further clinical variables for each subject, for example the subject classification according to a classification model disclosed herein. In other embodiments, the training data comprises data about the subject such as body weight, ethnicity, presence or absence of biomarkers, medication, etc.

In some embodiments, the training set includes a reference population of at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, or at least about 1000 subjects. In other embodiments, the training set includes more than 1000 subjects.

In some embodiments, the classification model comprises determining a (relative) weight for each methylation pattern and each clinical variable that is taken into account.

In some embodiments, the classification model uses data indicating the (relative) weight of each methylation pattern and each clinical variable for the determination of the risk of developing ADD.

In some embodiments, determining the risk score comprises correlating each of at least one of the methylation patterns and each of at least one clinical variable with its determined weight.

Classification models described herein can include different sets and combinations of methylation patterns and/or clinical variables. The classification model selects the methylation patterns and/or clinical variables according to the contribution or importance associated to each methylation pattern and/or clinical variable (i.e., determined weight). That is, the classification model is configured to combine the methylation pattern of sites which are correlated to a determined weight (e.g., 0.25, 0.5, 1, 2, 2.5, 5, etc.) and/or clinical variables which are correlated to a determined weight of at least 1 (e.g., 0.25, 0.5, 1, 2, 2.5, 5, etc.).

In some embodiments, the determined weight correlated to a methylation pattern or a clinical variable range from 0 to 100. Alternatively, the determined weight correlated to a methylation pattern, or a clinical variable is selected from 0 to 100. The value “0” corresponds to the lowest variable importance or lowest determined weight and the value “100” corresponds to the highest variable importance or highest determined weight. In a particular embodiment, the determined weight is selected from the group consisting of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

In some embodiments, the determined weight is at least 0.25. Particularly, the determined weight is selected from the group consisting of 0.25, 0.3, 0.35, 0.40, 0.45, 0.5, 0.55, 0.6, 0.65, 0.7, 0.75, 0.8, 0.85, 0.9, 0.95 and 1. In another embodiment, the determined weight is at least 1. Particularly, the determined weight is selected from the group consisting of 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 15, 20, 25, 30, 35, 40, 45, 50, 55, 60, 65, 70, 75, 80, 85, 90, 95 and 100. In another embodiment the determined weight is at least 2. Particularly, the determined weight is selected from the group consisting of 2, 2.25, 2.5, 2.75, 3, 3.25, 3.5, 3.75, 4, 4.25, 4.5, 4.75, 5, 6, 7, 8, 9, 10, 15, 20, 25, 30, 35, 40, 45, 50, 55, 60, 65, 70, 75, 80, 85, 90, 95 and 100. In another embodiment, the determined weight is selected from the group consisting of at least 1, at least 2, at least 2.5, at least 3, at least 3.5, at least 4, at least 4.5, at least 5, at least 7.5, at least 10, at least 15, at least 20, at least 25, at least 50 and at least 75.

In some embodiments, determining the risk of developing ADD in a subject comprises considering the methylation pattern of sites which are correlated to a determined weight of at least 0.5. In another embodiment, determining the risk of developing ADD in a subject comprises combining the methylation pattern of sites which are correlated to a determined weight of at least 0.5.

In another embodiment, determining the risk of developing ADD in a subject comprises considering the clinical variables which are correlated to a determined weight of at least 0.5. In another embodiment, determining the risk of developing ADD in a subject comprises combining the clinical variables which are correlated to a determined weight of at least 0.5.

In another embodiment, determining the risk of developing ADD in a subject comprises combining the methylation pattern of sites which are correlated to a determined weight of at least 0.5 with clinical variables which are correlated to a determined weight of at least 0.5. Alternatively, determining the risk of developing ADD in a subject comprises combining or considering methylation patterns and/or clinical variables correlated with a determined weight of at least 0.5.

In some embodiments, determining the risk of developing ADD in a subject comprises considering the methylation pattern of sites which are correlated to a determined weight of at least 1. In another embodiment, determining the risk of developing ADD in a subject comprises combining the methylation pattern of sites which are correlated to a determined weight of at least 1.

In another embodiment, determining the risk of developing ADD in a subject comprises considering the clinical variables which are correlated to a determined weight of at least 1. In another embodiment, determining the risk of developing ADD in a subject comprises combining the clinical variables which are correlated to a determined weight of at least 1.

In another embodiment, determining the risk of developing ADD in a subject comprises combining the methylation pattern of sites which are correlated to a determined weight of at least 1 with clinical variables which are correlated to a determined weight of at least 1. Alternatively, determining the risk of developing ADD in a subject comprises combining or considering methylation patterns and/or clinical variables correlated with a determined weight of at least 1.

In some embodiments, determining the risk of developing ADD in a subject comprises considering the methylation pattern of sites which are correlated to a determined weight of at least 2. In another embodiment, determining the risk of developing ADD in a subject comprises combining the methylation pattern of sites which are correlated to a determined weight of at least 2.

In another embodiment, determining the risk of developing ADD in a subject comprises considering the clinical variables which are correlated to a determined weight of at least 2. In another embodiment, determining the risk of developing ADD in a subject comprises combining the clinical variables which are correlated to a determined weight of at least 2.

In another embodiment, determining the risk of developing ADD in a subject comprises combining the methylation pattern of sites which are correlated to a determined weight of at least 2 with clinical variables which are correlated to a determined weight of at least 2. Alternatively, determining the risk of developing ADD in a subject comprises combining or considering methylation patterns and/or clinical variables correlated with a determined weight of at least 2.

In some embodiments, determining the risk of developing ADD in a subject comprises considering the methylation pattern of sites which are correlated to a determined weight of at least 2.5. In another embodiment, determining the risk of developing ADD in a subject comprises combining the methylation pattern of sites which are correlated to a determined weight of at least 2.5.

In another embodiment, determining the risk of developing ADD in a subject comprises considering the clinical variables which are correlated to a determined weight of at least 2.5. In another embodiment, determining the risk of developing ADD in a subject comprises combining the clinical variables which are correlated to a determined weight of at least 2.5.

In another embodiment, determining the risk of developing ADD in a subject comprises combining the methylation pattern of sites which are correlated to a determined weight of at least 2.5 with clinical variables which are correlated to a determined weight of at least 2.5. Alternatively, determining the risk of developing ADD in a subject comprises combining or considering methylation patterns and/or clinical variables correlated with a determined weight of at least 2.5.

In some embodiments, determining the risk of developing ADD in a subject comprises considering the methylation pattern of sites which are correlated to a determined weight of at least 5. In another embodiment, determining the risk of developing ADD in a subject comprises combining the methylation pattern of sites which are correlated to a determined weight of at least 5.

In another embodiment, determining the risk of developing ADD in a subject comprises considering the clinical variables which are correlated to a determined weight of at least 5. In another embodiment, determining the risk of developing ADD in a subject comprises combining the clinical variables which are correlated to a determined weight of at least 5.

In another embodiment, determining the risk of developing ADD in a subject comprises combining the methylation pattern of sites which are correlated to a determined weight of at least 5 with clinical variables which are correlated to a determined weight of at least 5. Alternatively, determining the risk of developing ADD in a subject comprises combining or considering methylation patterns and/or clinical variables correlated with a determined weight of at least 5.

Alternatively, the methylation patterns and/or clinical variables to be combined or considered by the classification model for determining the risk of developing ADD are selected according to their determined weight. Particularly, the methylation patterns and/or clinical variables combined by the classification model are selected according to their determined weight and the determined weight is at least 1. In another particular embodiment, the methylation patterns and/or clinical variables combined by the classification model are selected according to their determined weight and the determined weight is at least 2. In some embodiments, the methylation patterns and/or the clinical variables combined by the classification model for determining the risk of developing ADD are correlated to a determined weight selected from the group consisting of at least 0.25, at least 0.5, at least 1, at least 2, at least 2.5, at least 3, at least 3.5, at least 4, at least 4.5, at least 5, at least 7.5, at least 10, at least 15, at least 20, at least 25, at least 50 and at least 75.

In some embodiments, the classification model is capable of classifying subjects into two categories: progression to ADD and non-progression to ADD. In some embodiments, the classification model calculates a risk score to each category, corresponding to the probability of the subject to be assigned each category. In some embodiments, the probabilities to each category add up to 1.

Hereinbefore, weights correlated to a methylation pattern or a clinical variable range from 0 to 100 or are selected from 0 to 100, i.e. a scale of 0 to 100 is used. For this scale, specific minimum values of weights have been defined herein. It should be clear however, that any other suitable scale can be used, e.g., a scale from 0 to 1 or a scale from 0 to 1000 or a scale from 0 to 20. With such a different scale, the minimum values of weights mentioned hereinbefore can be changed proportionally.

The classification models generated by the machine-learning methods disclosed herein (e.g., Random Forest) can be subsequently evaluated by determining the ability of the classifier to correctly classify each test subject. In some embodiments, the subjects of the training population used to derive the model are different from the subjects of the testing population used to test the model. As would be understood by a person skilled in the art, this allows one to predict the ability of the dataset used to train the classifier as to their ability to properly classify a subject whose output classification (e.g., dementia stage classification, i.e., progression to ADD or non-progression to ADD) is unknown.

In some embodiments, the classification model is evaluated for its ability to properly classify each subject of the training population using methods known to a person skilled in the art. For example, one can evaluate the classification model using cross validation, Leave One Out Cross Validation (LOOCV), n-fold cross validation, or jackknife analysis using standard statistical methods. In other embodiments, each classifier is evaluated for its ability to properly characterize those subjects of the training population which were not used to generate the classifier.

In some embodiments, the method used to evaluate the classification model for its ability to properly classify each subject of the training population is a method which evaluates the classification model's sensitivity (TPF, true positive fraction) and 1-specificity (FPF, false positive fraction). In one embodiment, the method used to test the classifier is Receiver Operating Characteristic (“ROC”) which provides several parameters to evaluate both the sensitivity and specificity of the result of the classification model generated, e.g., a model derived from the application of a Random Forest.

In some embodiments, the metrics used to evaluate the classification model for its ability to properly classify each subject of the training population comprise classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), Negative Predicted Value (NPV), or any combination thereof. In other embodiments, the metrics used to evaluate the classification model for its ability to properly classify each subject of the training population are classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), and Negative Predicted Value (NPV).

Another aspect of the invention relates to a computer-implemented method applicable to the methods described herein e.g., for obtaining a risk score of developing Alzheimer's disease dementia, the method comprising the following steps:
(a) providing or receiving as input data:
(1) the methylation pattern in the D-loop region, and/or in the ND1 gene of the mitochondrial DNA of a subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6, and optionally
(2) at least one clinical variable of the subject as described herein, and
(b) combining and weighting said methylation patterns and clinical variables using a classification model to obtain the risk score.

In an embodiment, the computer-implemented method of determining the risk of developing Alzheimer's disease dementia in a subject, comprises:
a) receiving data relating to a methylation pattern in (a) the D-loop region, and/or (b) the ND1 gene of mitochondrial DNA of the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6; and
b) determining a risk score indicative of the risk of developing Alzheimer's disease dementia, wherein the risk score is calculated using a classification model configured to combine the methylation pattern of one or more sites determined in step (a) with at least one clinical variable of the subject selected from the group consisting of: sex, Sum of Boxes Score, Mini-Mental State Exam, Positron Emission Tomography, presence or absence of β-amyloid protein, age, genotype levels of Apolipoprotein E, and recategorized genotype levels of Apolipoprotein E.

Particularly, the at least one clinical variable of the subject is selected from the group consisting of sex, age, APOE, alE4, PET, presence or absence of beta-amyloid protein, SOB and MMSE.

### Score

In some embodiments, the risk score is a score of 0-1, where 0 indicates lowest risk of developing ADD and 1 indicates highest risk of developing ADD in the subject. In a particular embodiment, a risk score of 0.5 or higher than 0.5 indicates that the subject is likely to develop ADD. Alternatively, a risk score of 0.5 or higher than 0.5 indicates that the subject is at high risk of developing ADD. In a particular embodiment, a risk score of 0.75 or higher than 0.75 indicates that the subject is at very high risk/will develop ADD. In some embodiments, a risk score lower than 0.5 indicates that the subject is unlikely to develop ADD. Alternatively, a risk score lower than 0.5 or higher indicates that the subject is at low risk of developing ADD. In a particular embodiment, a risk score of 0.25 or lower than 0.25 or higher indicates that the subject is at very low risk/will develop ADD.

In some embodiments, the risk score is a score of 0-100, where 0 indicates lowest risk of developing ADD and 100 indicates highest risk of developing ADD in the subject. In some embodiments, the risk score can be but is not limited to 1-2, 1-5, 1-10, 1-100, 0-10 and 0-100. It is understood that the risk score in the present invention can be any range of values which serve to correlate to the risk of developing ADD of a subject.

### Subjects

In some embodiments, the subject is suspected of having, is at risk of developing, or has been diagnosed with dementia. In some embodiments, the subject has a Clinical Dementia Rating Score of 0.5. Particularly, the subject is diagnosed with Mild Cognitive Impairment. In some embodiments, the subject has at least one symptom of mild dementia. Particularly, the subject has at least one symptom selected from the group consisting of mild memory loss, mild loss of attention capacity, difficulties with reasoning, planning or problem-solving, difficulties in language and declined visual depth perception.

“Clinical Dementia Rating” is a global summary obtained through a semi-structured interview of patients and informants, and the subject's cognitive status is rated in six domains of functioning including memory, orientation, judgment, and problem solving, community affairs, home and hobbies, and personal care. Each domain is assigned a score between 0 and 3, and the results are computed via an algorithm to obtain a CDR global score. The CDR global score ranges from 0 to 3 and allows for the grouping of subjects according to the severity of their dementia, wherein CDR=0 corresponds to absent cognitive impairment, CDR=0.5 is questionable or very mild dementia, CDR=1 is Mild Dementia, CDR=2 is Moderate Dementia, and CDR=3 is Severe Dementia.

Subjects with a CDR of 0.5 are diagnosed with Mild Cognitive Impairment (MCI), which is an early stage of memory loss or other cognitive ability loss, such as language or visual/spatial perception, in subjects who maintain the ability to independently perform most activities of daily living. Subjects with a CDR of 1 or over are considered to have already progressed to ADD.

Methylation Pattern and their Measurements

The methylation pattern of the mitochondrial DNA (mtDNA) sites described herein can be determined using any method in the art. For example, mtDNA methylation can be determined by treating samples with bisulfite and sequencing the treated samples.

In some embodiments, determining the methylation pattern comprises determining the methylation pattern in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6.

In some embodiments, determining the methylation pattern comprises determining in at least one site of (vi) the CHH sites in the ND1 region shown in Table 6 and at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4, and
(v) the CHH sites in the D-loop region shown in Table 5.

In some embodiments, determining the methylation pattern comprises determining the methylation pattern of at least one of the CHH sites of ND1 gene shown in Table 6. In some embodiments, determining the methylation pattern comprises determining the methylation pattern of at least ten of the CHH sites of ND1 gene shown in Table 6. In some embodiments, determining the methylation pattern comprises determining the methylation pattern of at least twenty-five of the CHH sites of ND1 gene shown in Table 6. In some embodiments, determining the methylation pattern comprises determining the methylation pattern of at least fifty of the CHH sites of ND1 gene shown in Table 6. In some embodiments, determining the methylation pattern comprises determining the methylation pattern of at least one hundred of the CHH sites of ND1 gene shown in Table 6. In some embodiments, determining the methylation pattern comprises determining the methylation pattern of all CHH sites in the ND1 gene shown in Table 6.

In some embodiments, determining the methylation pattern comprises determining the methylation pattern of at least one of the CHH sites of D-loop region shown in Table 5. In some embodiments, determining the methylation pattern comprises determining the methylation pattern of all CHH sites in the D-loop region shown in Table 5.

In some embodiments, determining the methylation pattern comprises determining the methylation pattern of at least one of the CHG sites of ND1 gene shown in Table 4. In some embodiments, determining the methylation pattern comprises determining the methylation pattern of all CHG sites in the ND1 gene shown in Table 4.

In some embodiments, determining the methylation pattern comprises determining the methylation pattern of at least one of the CHG sites of D-loop region shown in Table 3. In some embodiments, determining the methylation pattern comprises determining the methylation pattern of all CHG sites in the D-loop region shown in Table 3.

In some embodiments, determining the methylation pattern comprises determining the methylation pattern of at least one of the CpG sites of ND1 gene shown in Table 2. In some embodiments, determining the methylation pattern comprises determining the methylation pattern of all CpG sites in the ND1 gene shown in Table 2.

In some embodiments, determining the methylation pattern comprises determining the methylation pattern of at least one of the CpG sites of D-loop region shown in Table 1. In some embodiments, determining the methylation pattern comprises determining the methylation pattern of all CpG sites in the D-loop region shown in Table 1.

In some embodiments, determining the methylation pattern comprises determining methylation in all CpG, CHG and CHH sites of the D-loop region. In another embodiment, determining the methylation pattern comprises determining methylation in all CpG, CHG and CHH sites of the ND1 gene. In some embodiments, determining the methylation pattern comprises determining methylation in all CpG sites of the D-loop region and the ND1 gene. In some embodiments, determining the methylation pattern comprises determining methylation in all CHG sites of the D-loop region and the ND1 gene. In some embodiments, determining the methylation pattern comprises determining methylation in all CHH sites of the D-loop region and the ND1 gene. In some embodiments, determining the methylation pattern comprises determining the methylation pattern of all CpG, CHG and CHH sites of the D-loop region and ND1 gene.

The methylation pattern can be determined by any method known in the art. In some embodiments, the methylation pattern is determined by a technique selected from the group consisting of techniques based on bisulfite treatment, techniques based on biological identification and bisulfite-free and enzyme-free techniques.

In some embodiments, techniques based on bisulfite treatment include but are not limited to sequence-based analysis, analysis based on melting temperature and interaction-based analysis. In some embodiments, sequence-based analysis include but are not limited to bisulfite sequencing, methylation specific PCR (MS-PCR), methylation-sensitive single-nucleotide primer extension (Ms-SnuPE) and reduced representation bisulfite sequencing (RRBS). In some embodiments, analysis based on melting temperature include but are not limited to methylation-specific denaturing gradient gel electrophoresis (MS-DGGE), methylation-specific melting curve analysis (MS-MCA), methylation-specific high-resolution melting (MS-HRM). In some embodiments, interaction-based analysis include but are not limited to combined bisulfite-restriction analysis (COBRA) and Methylight assay.

In some embodiments, techniques based on biological identification include but are not limited to methods based on enzymatic digestion and bio-dependence reactions. In some embodiments, methods based on enzymatic digestion include but are not limited to Restriction-landmark genomic scanning (RLGS), online monitoring and Methylation sensitive restriction enzyme-PCR (MS-RE-PCR/Southern). In a particular embodiment, the bio-dependence reaction is Methyl capture using methyl-CpG binding domain (MBD) proteins.

In some embodiments, bisulfite-free and enzyme-free techniques include but are not limited to analysis based on direct oxidation and analysis based on the chemical decomposition of oxidation. In a particular embodiment, the analysis based on direct oxidation is choline chloride monolayer supported multiwalled carbon nanotubes (MWCNTs/Ch/GCE). In a particular embodiment, the analysis based on the chemical decomposition of oxidation is Na1O₄/LiBr.

In a particular embodiment, the methylation pattern is determined by a technique based on bisulfite treatment. Particularly, the methylation pattern is determined by a sequence-based analysis. More particularly, the methylation pattern is determined by bisulfite sequencing.

In some embodiments, the methylation pattern is determined by a sequencing technique selected from the group consisting of methylation specific PCR (MS-PCR), quantitative methylation specific polymerase chain reaction (qMSP), bisulfite sequencing, pyrosequencing, nanopore sequencing, MassArray, methylation-sensitive single-nucleotide primer extension (Ms-SnuPE), reduced representation bisulfite sequencing (RRBS), methylation-specific denaturing gradient gel electrophoresis (MS-DGGE), methylation-specific melting curve analysis (MS-MCA), methylation-specific high resolution melting (MS-HRM), combined bisulfite-restriction analysis (COBRA) and Methylight assay, methylation-specific restriction endonucleases analysis (MSRE), methylation-sensitive restriction enzyme sequencing (MRE-seq), Restriction-landmark genomic scanning (RLGS), methylated-DNA Immunoprecipitation MeDIP or MeDIP-seq, methyl capture using methyl-CpG binding domain (MBD) proteins, ChIP assays, methylation arrays, choline chloride monolayer supported multiwalled carbon nanotubes (MWCNTs/ch/CGE) and analysis based on the chemical decomposition of oxidation (NaIO₄/LiBr).

In a particular embodiment, the methylation pattern is determined by bisulfite sequencing. In some embodiments, bisulfite sequencing comprises a step of treating the sample with bisulfite and another step of sequencing the bisulfite-treated sample by PCR. In a particular embodiment, the bisulfite-treated sample is sequenced using kits which can be but are not limited to kits produced by Illumina. More particularly, the bisulfite-treated sample is sequenced using a kit selected from the group consisting of MiSeq reagent Kit v3-600-cycles (#MS-102-3003, Illumina), MiSeq reagent Kit v2-500-cycles (#MS-102-2003, Illumina) and MiSeq reagent Nano Kit v2-500 cycles (#MS-103-1003, Illumina).

Sequencing of samples can be performed using any known method in the art. Platforms of sequencing include but are not limited to Roche, Illumina, Life Technologies, Polonator, Helicos Bioscience, Pacific Biosciences, HTG Molecular Diagnostic, Singular Genomics, Element Biosciences, Oxford Nanopore and Nanostring Technology.

In some embodiments, determining the methylation pattern comprises a step of library quantification. In some embodiments, library quantification is performed using a fluorometric quantification method. Alternatively, quantification of the methylation pattern is determined using fluorescence. In a particular embodiment, the fluorometric quantification method is characterized in using kits comprising dsDNA binding dyes. In a particular embodiment, library quantification is performed using the Qubit® 3.0 Fluorometer produced by Thermo Fisher Scientific and kits produced by Thermo Fisher Scientific. More particularly, library quantification is performed using a kit selected from the group consisting of the Qubit™ dsDNA HS Assay Kit (#Q32854, Thermo Fisher Scientific and Qubit™ dsDNA BR Assay Kit and #Q32850, Thermo Fisher Scientific). More particularly, library quantification is performed using kits selected from the group consisting of the Qubit™ dsDNA Assay Kits (#Q32854, Thermo Fisher Scientific and Qubit™ dsDNA BR Assay Kit and #Q32850, Thermo Fisher Scientific).

It is understood that library quantification using a fluorometric quantification method in the present invention may be performed using fluorometers and kits comprising dsDNA binding dyes from other brands, beyond Thermo Fisher Scientific. An example of another suitable fluorometer would be but is not limited to QFX Fluorometer produced by DeNovix and Quantus™ Fluorometer produced by Promega. An example of other suitable dsDNA Fluorescence kits are but are not limited to QuantiFluor® Dye Systems and QuantiFluor® dsDNA produced by Promega. Further, QFX Fluorometer from DeNovix works with any of its own DeNovix dsDNA Fluorescence Quantification Kits and other common commercially available assays.

In the examples of the present invention, the analysis to compare methylation levels between in each methylation site is conducted using DSS (Dispersion Shrinkage for Sequencing data) Bioconductor package. In other embodiments, any other adequate methodology known in the art may be used. In some embodiments, the analysis is performed using beta-binominal based models. In some embodiments, the analysis is performed using non-beta-binomina based models.

In the examples of the present invention, the threshold p-value to establish differential methylation is 0.05. In other embodiments, the threshold p-value is stablished at 0.25. In other embodiments, the threshold p-value is stablished at 0.2. In other embodiments, the threshold p-value is stablished at 0.15. In other embodiments, the threshold p-value is stablished at 0.1. In other embodiments, the threshold p-value is stablished at 0.09. In other embodiments, the threshold p-value is stablished at 0.08. In other embodiments, the threshold p-value is stablished at 0.07. In other embodiments, the threshold p-value is stablished at 0.06. In other embodiments, the threshold p-value is stablished at 0.05. In other embodiments, the threshold p-value is stablished at 0.04. In other embodiments, the threshold p-value is stablished at 0.03. In other embodiments, the threshold p-value is stablished at 0.02. In other embodiments, the threshold p-value is stablished at 0.01.

In another aspect, the invention relates to a methylation site panel comprising at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6;
   for use in identifying a human subject at risk of developing ADD using a classification model configured to combine a methylation pattern in (a) the D-loop region, and/or (b) the ND1 gene of mitochondrial DNA from a sample obtained from the subject, with at least one clinical variable of the subject, as described herein.

### Clinical Variables

The classification model and methods described herein, e.g., the method to determine the risk of developing ADD in a subject, use input data comprising clinical variables of the subject. Further, the classification model is trained with a training dataset which comprises clinical variables associated to a plurality of subjects.

Clinical variables can include any clinical variable known in the art. In some embodiments, the method comprises combining the methylation patterns described herein with at least one clinical variable selected from the group consisting of Sum of Boxes Score (SOB), Mini-Mental State Exam (MMSE), Positron Emission Tomography (PET), presence or absence of β-amyloid protein, sex, age, genotype levels of Apolipoprotein E (APOE), recategorized genotype levels of Apolipoprotein (alE4), β-amyloid-42 protein, β-amyloid-40 protein, tau-T protein, tau-P protein, glial fibrillary acidic protein (GFAP), chitinase-3-like protein 1 (YKL-40), p53 and neurofilament light (NfL).

In some embodiments, the method comprises combining the methylation patterns described herein with at least one clinical variable of the subject selected from the group consisting of SOB, MMSE, presence or absence of β-amyloid protein, sex, age, APOE, alE4, Aβ-40, Aβ-42, tau-T and tau-P. In some embodiments, the method comprises combining the methylation patterns described herein with at least one clinical variable of the subject selected from the group consisting of SOB, MMSE, PET, sex, age, APOE, alE4, Aβ-42, tau-T and tau-P.

In another embodiment, the at least one clinical variable of the subject is selected from the group consisting of sex, SOB, MMSE, PET, presence or absence of β-amyloid protein, age, APOE, and alE4.

In a particular embodiment, the at least one clinical variable of the subject is selected from the group consisting of SOB, MMSE, PET, sex, age, APOE, and alE4. In another embodiment, the at least one clinical variable of the subject is selected from the group consisting of SOB, MMSE, sex, age, APOE, and alE4. In another embodiment, the at least one clinical variable of the subject is selected from the group consisting of SOB, MMSE, sex, and age. In another embodiment, the at least one clinical variable is presence or absence of β-amyloid protein. In a particular embodiment, the at least one clinical variable is SOB. In another embodiment, the at least one clinical variable is MMSE. In another embodiment, the clinical variables are at least SOB and MMSE. In another embodiment, the at least one clinical variable is PET, particularly β-amyloid-PET.

“Sex” is a categorical variable comprising two possible categories: Female or Male.

“Age” is a numerical variable which corresponds to the years of the subject.

“APOE” is a categorical variable comprising categories which correspond to the different genotype levels of Apolipoprotein E: E2.E2, E2.E3, E2.E4, E3.E3, E3.E4 and E4.E4.

“alE4” is a categorical variable corresponding to the recategorization of APOE genotype levels into the following categories: 0 (comprising E2.E2, E2.E3 and E3.E3), 1 (comprising E2.E4 and E3.E4) and 2 (comprising E4.E4).

“SOB” is a numerical variable, which refers to “Sum of Boxes Score”, a score ranging from 0 to 18 obtained by summing each of the domain box scores described above for the calculation of CDR global score.

“MMSE” is a numerical variable, which refers to “Mini-Mental State Exam”, an evaluation of five main items: orientation, fixation, concentration and calculation, memory and language, and construction with an output score between 1 and 30.

“PET” refers to Positron Emission Tomography, which is a type of nuclear medicine procedure that measures metabolic activity of the cells of body tissues. PET can be performed with different types of tracers, each of them used for a specific purpose, study or detection. For example, PET can measure glucose levels, beta-amyloid plaques or tau protein. FDG-PET refers to a PET designed to detect de glucose and consequently analyze metabolic activity of tissues or body sites. In another example, PET is used to determine the presence of beta-amyloid plaques in the brain. In an embodiment, PET is a categorical variable which refers to the presence or absence of β-amyloid determined via Positron Emission Tomography.

“Aβ-42” is a categorical variable which refers to the presence or absence of protein β-amyloid-42 (Aβ-42) in a sample of cerebrospinal fluid or any other adequate biological sample or fluid such as blood, or via radio imaging techniques (e.g., PET).

“Aβ-40” is a categorical variable which refers to the presence or absence of protein β-amyloid-40 (Aβ-40) in a sample of cerebrospinal fluid or any other adequate biological sample or fluid such as blood or via radio imaging techniques (e.g., PET).

“tau-T” is a categorical variable which refers to the presence or absence of protein Tau in a sample of cerebrospinal fluid or any other adequate biological sample or fluid such as blood or via radio imaging techniques.

“tau-P” is a categorical variable which refers to the presence or absence of protein Tau phosphorylated in a sample of cerebrospinal fluid or any other adequate biological sample or fluid such as blood or via radio imaging techniques. Protein Tau-P can be phosphorylated in one or more positions such as 181, 217, 231 (i.e., p-tau-181, p-tau-217, p-tau-231), among other options.

“GFAP” is a categorical variable which refers to the presence or absence of glial fibrillary acidic protein in a sample of cerebrospinal fluid or any other adequate biological sample or fluid such as blood.

“YKL-40” is a categorical variable which refers to the presence or absence of chitinase-3-like protein in a sample of cerebrospinal fluid or any other adequate biological sample or fluid such as blood.

“p53” is a gene that codifies for the tumor protein p53, which can adopt multiple structural and functional states, including altered conformation states which can contribute to the development of neurodegenerative diseases such as AD. Thus, “p53” is a categorical variable which refers to detection of conformational variants of p53 related to the development of Alzheimer's disease.

“NfL” is a categorical variable which refers to the presence or absence of neurofilament light (NfL) in plasma, or any other adequate biological sample or fluid.

In some embodiments, clinical variables include the determination of the presence or absence of β-amyloid protein. The presence or absence of β-amyloid protein can be determined using different techniques including PET scan, analysis of cerebrospinal fluid (CSF), retinal screening and blood test. Therefore, in some embodiments, clinical variables comprise the presence or absence of β-amyloid protein. Particularly, clinical variables comprise the presence or absence of β-amyloid protein determined by PET scan, CSF analysis, blood test and/or retinal screening. In a particular embodiment, clinical variables include the presence or absence of β-amyloid protein determined by PET scan and/or CSF analysis.

As described above, clinical variables can include biomarkers known in the art, as well as other neuropsychological tests and radio imaging techniques known in the art. In some embodiments, clinical variables include the presence, absence or the levels of biomarkers known in the art (e.g., Aβ-42). In some embodiments, clinical variables include the presence or absence of biomarkers known in the art.

In some embodiments, biomarkers can be measured in any sample or fluid from the subject. In a particular embodiment, biomarkers are measured in cerebrospinal fluid (CSF) samples and/or blood samples. Particularly, biomarkers are measured in cerebrospinal fluid (CSF) samples and/or blood samples and are selected from the group consisting of Aβ-40, Aβ-42, neurofilament light (NfL), tau-T, tau-P (e.g., p-tau-181, p-tau-217, p-tau-231, etc.), GFAP, p-53, and/or YKL-40.

In other embodiments, biomarkers can be detected using radio imaging techniques, and particularly positron emission tomography (PET). In another embodiment, biomarkers are detected using radio imaging techniques and biomarkers are selected from the group consisting of β-amyloid protein and tau protein, and particularly, Aβ-40, Aβ-42, tau-T and tau-P (e.g., p-tau-181, p-tau-217, p-tau-231, etc.). In some embodiments, clinical variables include data obtained through invasive methods, such as PET or Aβ-40, Aβ-42, tau-T, tau-P and GFAP, which may require the analysis of a cerebrospinal fluid sample. In other embodiments, clinical variables only include data obtained through non-invasive methods.

In some embodiments, clinical variables can include other neuroimaging techniques. In other embodiments, clinical variables can derive from medical imaging such as PET or magnetic resonance imaging (MRI). In some embodiments, PET is used to measure glucose, beta-amyloid protein and/or tau protein. In a particular embodiment, PET is used to measure beta-amyloid protein.

In other embodiments, clinical variables include a retinal screening. Particularly, the retinal screening determines the presence or absence of biomarkers. More particularly, the retinal screening determines the presence or absence of β-amyloid protein.

In some embodiments, clinical variables can include a variable relating to a current treatment or medication of the subject, particularly for dementia or dementia-related symptoms.

In an embodiment, the method of determining the risk of developing Alzheimer's disease dementia in a subject, comprises:
a) determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6; and
b) determining a risk score indicative of the risk of developing Alzheimer's disease dementia, wherein the risk score is calculated using a classification model configured to combine the methylation pattern of one or more sites determined in step (a) with at least one clinical variable of the subject selected from the group consisting of: sex, Sum of Boxes Score, Mini-Mental State Exam, Positron Emission Tomography, presence or absence of β-amyloid protein, age, genotype levels of Apolipoprotein E, and recategorized genotype levels of Apolipoprotein E.

### Samples and Samples Processing

The terms “biological sample” or “sample” as used herein refers to biological material isolated from a subject. The biological sample can contain any biological material suitable for determining methylation patterns, e.g., by treating and sequencing nucleic acids.

In some embodiments, the sample is selected from a biofluid or biopsy of a solid tissue. Particularly, the sample is selected from the group consisting of blood, plasma, saliva, cerebrospinal fluid, brain sample, skin sample and urine. Particularly, the sample is blood, particularly peripheral blood.

The source of the sample can be solid tissue, e.g., from a fresh, frozen and/or preserved organ, tissue sample, biopsy, or aspirate. In some embodiments, the sample is a cell-free sample, e.g., comprising cell-free nucleic acids (e.g., DNA or RNA). A sample can, in some embodiments, comprise compounds that are not naturally intermixed with the tissue in nature such as preservatives, anticoagulants, buffers, fixatives, nutrients, antibiotics or the like.

In some embodiments, the method comprises obtaining the sample. In a particular embodiment, the sample is blood or plasma, and the sample is extracted by using a needle. In another particular embodiment, the sample is saliva, and the sample is obtained using a method selected from the group consisting of draining method, spitting method, suction method and swab method. In some embodiments, the sample can be obtained, e.g., from surgical material or from biopsy. In some embodiments, the biopsy can be archival tissue from a previous line of therapy. In some embodiments, the biopsy can be from tissue that is therapy naïve.

In some embodiments, the sample is frozen or preserved. In some embodiments, the sample is preserved as a frozen sample or as formalin-, formaldehyde-, or paraformaldehyde-fixed paraffin-embedded (FFPE) tissue preparation. For example, the sample can be embedded in a matrix, e.g., an FFPE block or a frozen sample. In some embodiments, a sample can comprise bone marrow; aspirates; scrapings; bone marrow specimens; tissue biopsy specimens; surgical specimens; etc. In some embodiments, a sample is or comprises cells obtained from an individual, e.g., from an individual from whom the sample is obtained.

In some embodiments, samples are fresh samples (or non-archival samples) or archival samples. As used herein, the terms “fresh sample,” “non-archival sample,” and grammatical variants thereof refer to a sample which has been processed before a predetermined period of time, e.g., one week, after extraction from a subject. In some embodiments, a fresh sample has not been frozen. In some embodiments, a fresh sample has not been fixed. In some embodiments, a fresh sample has been stored for less than about two weeks, less than about one week, or less than six, five, four, three, or two days before processing. As used herein, the term “archival sample” and grammatical variants thereof refers to a sample which has been processed after a predetermined period of time, e.g., a week, after extraction from a subject. In some embodiments, an archival sample has been frozen. In some embodiments, an archival sample has been fixed. In some embodiments, an archival sample has a known diagnostic and/or a treatment history. In some embodiments, an archival sample has been stored for at least one week, at least one month, at least six months, or at least one year, before processing.

In another aspect, the invention relates to an enriched sample obtained from a subject at risk of developing ADD comprising mitochondrial DNA suitable for use in determining a methylation pattern in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6.

### Oligonucleotides and Kits

### Oligonucleotides

As discussed, in some embodiments, the methylation pattern is determined using at least one oligonucleotide capable of specifically hybridizing a mitochondrial DNA sequence comprising the D-loop region or the ND1 gene. Particularly, oligonucleotides are capable of specifically hybridizing under high stringency conditions.

The sequence of interest may refer to the reference sequence or the sequence resulting from certain modification treatment, e.g., bisulfite treatment wherein unmethylated cytosines are modified to uracil. In some embodiments, the oligonucleotide hybridizes the reference mitochondrial DNA sequence comprising the D-loop region or the ND1 gene. In other embodiments, the oligonucleotide hybridizes a modified mitochondrial DNA sequence comprising the D-loop region or the ND1 gene. In a particular embodiment, the modified mitochondrial DNA sequence has been modified by bisulfite treatment. In a more particular embodiment, the modified mitochondrial DNA sequence has been modified by bisulfite treatment, wherein non-methylated cytosines have been modified to uracil.

In some embodiments, the methylation pattern is determined using at least one oligonucleotide/primer capable of specifically hybridizing with a mitochondrial DNA sequence comprising a methylation site selected from the group consisting of:
i) the CpG sites in the D-loop region shown in Table 1,
ii) the CpG sites of the ND1 gene shown in Table 2,
iii) the CHG sites in the D-loop region shown in Table 3,
iv) the CHG sites in the ND1 gene shown in Table 4,
v) the CHH sites in the D-loop region shown in Table 5, and
vi) the CHH sites in the ND1 region shown in Table 6.

In some embodiments, the oligonucleotide is capable of specifically hybridizing with a mitochondrial DNA sequence comprising the D-loop region. In a particular embodiment, the oligonucleotide is capable of specifically hybridizing with a mitochondrial DNA sequence comprising at least one site selected from the group consisting of: the CpG sites in the D-loop region shown in Table 1, the CHG sites in the D-loop region shown in Table 3 and the CHH sites in the D-loop region shown in Table 5. More particularly, the oligonucleotide is capable of specifically hybridizing with a mitochondrial DNA sequence comprising all sites of the CpG sites in the D-loop region shown in Table 1, the CHG sites in the D-loop region shown in Table 3 and the CHH sites in the D-loop region shown in Table 5.

In some embodiments, the oligonucleotide is capable of specifically hybridizing with a mitochondrial DNA sequence comprising the ND1 gene. In a particular embodiment, the oligonucleotide is capable of specifically hybridizing with a mitochondrial DNA sequence comprising at least one site selected from the group consisting of: the CpG sites in the ND1 gene shown in Table 2, the CHG sites in the ND1 gene shown in Table 4 and the CHH sites in the ND1 gene shown in Table 4. More particularly, the oligonucleotide is capable of specifically hybridizing with a mitochondrial DNA sequence comprising all sites of the CpG sites in the ND1 gene shown in Table 2, the CHG sites in the ND1 gene shown in Table 4 and the CHH sites in the ND1 gene shown in Table 4.

In an embodiment, the oligonucleotides are DNA sequences.

Inventors herein designed primers which included the least number of cytosines. In some embodiments, the primers are degenerated to cover all possible methylated and no methylated scenarios due to the uncertain C/U conversion of the few cytosines residues included in the sequences. These primers are a mixture of oligonucleotide sequences which contain several possible nucleotide bases at certain position. Consequently, the probability to detect mitochondrial methylation is higher. The forward degenerated primers include Y, which refers to either C or T (Y=C/T), in any position wherein the reference sequence is a C. Further, as known in the art, reverse primers do not correspond to the reference sequence, but to the reversed complementary of the reference sequence. Therefore, the reverse primers do not include C sites of the reference sequence, but their complementary G sites. Thus, reverse degenerated primers will include R, which refers to either G or A (R=A/G), in any position wherein the reference sequence is a C, or wherein the complementary sequence includes a G.

In a particular embodiment, the methylation pattern is determined using at least one oligonucleotide selected from the group consisting of SEQ ID NO: 1, SEQ ID NO: 2, SEQ ID NO: 3 and SEQ ID NO: 4. Oligonucleotides can comprise additional nucleotides in their ends to be adequate for using in e.g., sequencing (i.e., sequencing adapters). In a particular embodiment, the methylation pattern is determined using at least one oligonucleotide with a length between 15 and 100 nucleotides and comprising a sequence selected from the group consisting of SEQ ID NO: 1, SEQ ID NO: 2, SEQ ID NO: 3 and SEQ ID NO: 4.

In a more particular embodiment, the methylation pattern is determined using oligonucleotides with a length between 15 and 100 nucleotides and comprising sequences SEQ ID NO: 1, SEQ ID NO: 2, SEQ ID NO: 3 and SEQ ID NO: 4. In a particular embodiment, the methylation pattern is determined using the oligonucleotides SEQ ID NO: 1, SEQ ID NO: 2, SEQ ID NO: 3 and SEQ ID NO: 4.

An aspect of the invention relates to a oligonucleotide with a length between 15 and 100 nucleotides and comprising a sequence selected from the group consisting of SEQ ID NO: 1, SEQ ID NO: 2, SEQ ID NO: 3, and SEQ ID NO: 4. In a particular embodiment, the oligonucleotide is selected from the group consisting of SEQ ID NO: 1, SEQ ID NO: 2, SEQ ID NO: 3, and SEQ ID NO: 4. As said, in an embodiment, the nucleic acid sequence comprises sequencing adapters at the ends of SEQ ID NO: 1, 2, 3 and/or 4. In a particular embodiment, the oligonucleotide is SEQ ID NO: 1. In another particular embodiment, the oligonucleotide is SEQ ID NO: 2. In another particular embodiment, the oligonucleotide is SEQ ID NO: 3. In another particular embodiment, the oligonucleotide is SEQ ID NO: 4.

An aspect of the invention relates to the use of a oligonucleotide with a length between 15 and 100 nucleotides and comprising a sequence selected from the group consisting of SEQ ID NO: 1, SEQ ID NO: 2, SEQ ID NO: 3, and SEQ ID NO: 4 for the determination of a methylation pattern of mitochondrial DNA.

In a particular embodiment, the invention relates to the use of an oligonucleotide with a length between and 100 nucleotides and comprising sequence SEQ ID NO: 1 (and particularly consisting of SEQ ID NO: 1) for the determination of a methylation pattern of a mitochondrial DNA sequence comprising the D-loop region. In another embodiment, the invention relates to the use of an oligonucleotide with a length between 15 and 100 nucleotides and comprising sequence SEQ ID NO: 2 (and particularly consisting of SEQ ID NO: 2) for the determination of a methylation pattern of a mitochondrial DNA sequence comprising the D-loop region. In another embodiment, the invention relates to the use of an oligonucleotide with a length between 15 and 100 nucleotides and comprising sequence SEQ ID NO: 3 (and particularly consisting of SEQ ID NO: 3) for the determination of a methylation pattern of a mitochondrial DNA sequence comprising the ND1 gene. In another embodiment, the invention relates to the use of an oligonucleotide with a length between 15 and 100 nucleotides and comprising sequence SEQ ID NO: 4 (and particularly consisting of SEQ ID NO: 4) for the determination of a methylation pattern of a mitochondrial DNA sequence comprising the ND1 gene.

In some embodiments, the invention relates to the use of an oligonucleotide with a length between 15 and 100 nucleotides and comprising a sequence selected from the group consisting of SEQ ID NO: 1, SEQ ID NO: 2, SEQ ID NO: 3, and SEQ ID NO: 4 for the determination of a methylation pattern of mitochondrial DNA to determine the risk of developing ADD in a subject.

### Kits

In an aspect, the invention relates to a kit comprising at least one oligonucleotide capable of specifically hybridizing with a mitochondrial DNA sequence comprising the D-loop region or the ND1 gene. In some embodiments, the kit comprises the oligonucleotides as previously defined.

Particularly, the kit comprises at least one oligonucleotide with a length between 15 and 100 nucleotides and comprising a sequence selected from the group consisting of SEQ ID NO: 1, SEQ ID NO: 2, SEQ ID NO: 3, and SEQ ID NO: 4. In a particular embodiment, the invention relates to a kit comprising oligonucleotides with a length between 15 and 100 nucleotides, comprising the nucleic acid sequences SEQ ID NO: 1 and SEQ ID NO: 2. In another embodiment, the invention relates to a kit comprising oligonucleotides with a length between 15 and 100 nucleotides, comprising the nucleic acid sequences SEQ ID NO: 3 and SEQ ID NO: 4. In a particular embodiment, the invention relates to a kit comprising oligonucleotides with a length between 15 and 100 nucleotide, and comprising the nucleic acid sequences SEQ ID NO: 1, SEQ ID NO: 2, SEQ ID NO: 3 and SEQ ID NO: 4.

In a particular embodiment, the kit comprises oligonucleotides with a length between 15 and 100 nucleotides and comprising SEQ ID NO: 1 and SEQ ID NO: 2 for the determination of a methylation pattern of a mitochondrial DNA sequence comprising the D-loop region. In another embodiment, the kit comprises oligonucleotides with a length between 15 and 100 nucleotides and comprising SEQ ID NO: 3 and SEQ ID NO: 4 for the determination of a methylation pattern of a mitochondrial DNA sequence comprising the ND1 gene.

In an aspect, the invention relates to the use of the kits as described above for the determination of a methylation pattern of mitochondrial DNA. In another aspect, the invention relates to the use of the kits as defined above, for the determination of a methylation pattern of mitochondrial DNA to determine the risk of developing ADD in a subject. In another aspect, the invention relates to the use of the kits as defined above, following the methods as described herein.

Such kits can comprise containers, each with one or more of the various reagents (e.g., in concentrated form) utilized in the method, including, e.g., one or more oligonucleotides (e.g., oligonucleotides with SEQ ID NO: 1-4 provided herein). The kit can also provide reagents, buffers, and/or instrumentation to support the practice of the methods provided herein.

A kit provided according to the invention can also comprise brochures or instructions describing the methods disclosed herein or their practical application to determine the risk of developing ADD in a subject. Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, the kit is an Illumina sequencing kit. More particularly, the kit is selected from the group consisting of MiSeq reagent Kit v3-600-cycles (#MS-102-3003, Illumina), MiSeq reagent Kit v2-500-cycles (#MS-102-2003, Illumina) and MiSeq reagent Nano Kit v2-500 cycles (#MS-103-1003, Illumina).

In another aspect, the invention is related to a kit comprising:
a) reagents to determine a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6; and,
b) optionally instructions to use the reagents.

### Companion Diagnostic Systems

The methods disclosed herein can be provided as a companion diagnostic, e.g., available via a web server, to inform the clinician or patient about potential treatment choices or for the selection of patients for a clinical trial. The methods disclosed herein can comprise collecting or otherwise obtaining a biological sample and performing an analytical method disclosed herein to determine the risk of developing ADD in a subject.

In an aspect of the present invention, a computing system is provided which comprises suitable means for carrying out any of the computer-implemented methods described herein.

At least some embodiments of the methods described herein, due to the complexity of the calculations involved can be implemented with the use of a computer. In some embodiments the computer system comprises hardware elements that are electrically coupled via bus, including a processor, input device, output device, storage device, computer-readable storage media reader, communications system, processing acceleration (e.g., DSP or special-purpose processors), and/or memory. The computer-readable storage media reader can be further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource.

A single architecture might be utilized to implement one or more servers that can be further configured in accordance with currently desirable protocols, protocol variations, extensions, etc. However, it will be apparent to those skilled in the art that embodiments can well be utilized in accordance with more specific application requirements. Customized hardware might also be utilized and/or particular elements might be implemented in hardware, software, firmware or combinations thereof. Further, while connection to other computing devices such as network input/output devices (not shown) can be employed, it is to be understood that wired, wireless, modem, and/or other connection or connections to other computing devices might also be utilized.

In one embodiment, the system further comprises one or more devices for providing input data to the one or more processors. The system further comprises a memory for storing a dataset of ranked data elements. In another embodiment, the device for providing input data comprises a detector for detecting the characteristic of the data element, e.g., such as a fluorescent plate reader, mass spectrometer, or gene chip reader.

The system additionally can comprise a database management system. User requests or queries can be formatted in an appropriate language understood by the database management system that processes the query to extract the relevant information from the database of training sets. The system can be connectable to a network to which a network server and one or more clients are connected. The network can be a local area network (LAN) or a wide area network (WAN), as is known in the art. Particularly, the server includes the hardware necessary for running computer program products (e.g., software) to access database data for processing user requests. The system can be in communication with an input device for providing data regarding data elements to the system (e.g., methylation patterns).

In a further aspect, the invention is directed to a computer program product comprising instructions which, when the program is executed by a computer, cause the computer to carry out any of the computer-implemented methods described herein.

Some embodiments described herein can be implemented so as to include a computer program product. A computer program product can include a computer readable medium having computer readable program code embodied in the medium for causing an application program to execute on a computer with a database. As used herein, a “computer program product” refers to an organized set of instructions in the form of natural or programming language statements that are contained on a physical media of any nature (e.g., written, electronic, magnetic, optical or otherwise) and that can be used with a computer or other automated data processing system. Such programming language statements, when executed by a computer or data processing system, cause the computer or data processing system to act in accordance with the particular content of the statements.

In some embodiments, the invention is directed to a computer program product which includes a computer readable medium embodying program code executable by a processor of a computing device or system, the program code comprising code that executes a classification model for e.g., identifying a human subject at risk of developing ADD (or other methods described herein) configured to combine a methylation pattern with at least one clinical variable as described herein; wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6.

In an embodiment, the invention relates to a computer program product which includes a computer readable medium embodying program code executable by a processor of a computing device or system, the program code comprising code that executes a classification model e.g., for identifying a human subject risk of developing ADD (or other methods described herein), wherein the model is configured to identify the human subject as being at risk of developing ADD, wherein the classification model is configured to combine a methylation pattern with at least one clinical variable of the subject, as described herein; wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6.

Computer program products include without limitation: programs in source and object code and/or test or data libraries embedded in a computer readable medium. Furthermore, the computer program product that enables a computer system or data processing equipment device to act in pre-selected ways can be provided in a number of forms, including, but not limited to, original source code, assembly code, object code, machine language, encrypted or compressed versions of the foregoing and any and all equivalents. In one aspect, a computer program product is provided to implement the treatment, diagnostic, methods disclosed herein, for example, to determine whether to administer a certain therapy based on the obtained score.

The computer program product includes a computer readable medium embodying program code executable by a processor of a computing device or system, the program code comprising:
(a) code that retrieves data attributed to a biological sample from a subject, wherein the data comprises the methylation patterns corresponding to methylation sites of Tables 1-6 in the biological sample or wherein the methylation patterns can be derived from the data. These values are combined with values corresponding to clinical variables; and,
(b) code that executes a classification method that indicates, e.g., whether to administer a therapeutic agent to a patient in need thereof based on the obtained scores.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description.

Furthermore, some embodiments can be code stored in a computer-readable memory of virtually any kind including, without limitation, RAM, ROM, magnetic media, optical media, or magneto-optical media. Even more generally, some embodiments could be implemented in software, or in hardware, or any combination thereof including, but not limited to, software running on a general purpose processor, microcode, PLAs, or ASICs.

It is also envisioned that some embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### Performance of the Methods

In some embodiments, samples can, e.g., be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the determination of the methylation patterns disclosed herein; the application of the classification model; the determination of scores; treatment decisions; clinical trials inclusion decisions; or combinations thereof, can be performed by one or more healthcare providers, healthcare benefits providers, and/or clinical laboratories.

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, diagnosis, assessment, maintenance, therapy, medication, and/or advice relating to all, or any portion of, a patient's state of health, including but not limited to general medical, specialized medical, surgical, and/or any other type of treatment, diagnosis, assessment, maintenance, therapy, medication and/or advice. A healthcare provider also refers herein to pharmaceutical companies or its providers/intermediates (e.g., CRO) involved in the development of clinical trials.

As used herein, the term “clinical laboratory” refers to a facility for the examination or processing of materials derived from a subject. These examinations can also include procedures to collect or otherwise obtain a sample, prepare, determine, measure, or otherwise describe the presence or absence of various substances in the body of a subject or a sample obtained from the body of a subject (e.g., methylation patterns of mtDNA or biomarkers used herein as clinical variables) Examinations can also include procedures such as medical imaging procedures (e.g., PET, MRI) to obtain clinical variables data.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

A healthcare provider can implement or instruct another healthcare provider or patient to perform the following actions: obtain a sample/clinical variables, process a sample/clinical variables, submit a sample/clinical variables, receive a sample/clinical variables, transfer a sample/clinical variables, analyze or measure a sample/clinical variables (e.g. to obtain the methylation patterns), quantify a sample/clinical variables, provide the results obtained after analyzing/measuring/quantifying a sample/clinical variables, receive the results obtained after analyzing/measuring/quantifying a sample/clinical variables, apply the classification model, score the results obtained after analyzing/measuring/quantifying one or more samples/clinical variables, provide the score from one or more samples, obtain the score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some embodiments, a healthcare benefits provider can authorize or deny, e.g., collection of a sample/clinical variables, processing of a sample/clinical variables, submission of a sample/clinical variables, receipt of a sample/clinical variables, transfer of a sample/clinical variables, analysis or measurement a sample/clinical variables (e.g. to obtain the methylation patterns), quantification of a sample/clinical variables, apply the classification model, provision of results obtained after analyzing/measuring/quantifying a sample/clinical variables, transfer of results obtained after analyzing/measuring/quantifying a sample/clinical variables, scoring of results obtained after analyzing/measuring/quantifying one or more samples/clinical variables, transfer of the score from one or more samples/clinical variables, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent. In addition, a healthcare benefits provides can, e.g., authorize or deny the prescription of a therapy, authorize or deny coverage for therapy, authorize or deny reimbursement for the cost of therapy, determine or deny eligibility for therapy, etc.

In some embodiments, a clinical laboratory can, for example, collect or obtain a sample/clinical variables, process a sample/clinical variables, submit a sample/clinical variables, receive a sample/clinical variables, transfer a sample/clinical variables, analyze or measure a sample/clinical variables (e.g. to obtain the methylation patterns), quantify a sample/clinical variables, apply the classification model, provide the results obtained after analyzing/measuring/quantifying a sample/clinical variables, receive the results obtained after analyzing/measuring/quantifying a sample/clinical variables, score the results obtained after analyzing/measuring/quantifying one or more samples/clinical variables, provide the score from one or more samples/clinical variables, obtain the score from one or more samples/clinical variables, or other related activities.

In some embodiments, the sample/clinical variables can be obtained by a healthcare professional treating or diagnosing the patient, by a healthcare provider or by a clinical laboratory. Measurements of the sample (e.g., by using a particular assay described herein) and obtaining clinical variables (e.g., by medical imaging techniques) can be performed by a healthcare provider or a clinical laboratory, being the same or different from the ones that obtained the sample/clinical variables. The classification model can be applied by the healthcare provider or a different healthcare provider or by the clinical laboratory. The score and results obtained are finally sent to the first healthcare professional treating or diagnosing the patient or to the healthcare provider. Thus, in some embodiments, the healthcare provider or the clinical laboratory can advise the healthcare professional/provider about diagnosis or as to whether the patient can benefit from treatment. In some embodiments, the healthcare provider is a pharmaceutical company or one of its providers/intermediates (e.g., CRO) involved in the development of clinical trials. All the steps described herein can be performed by the pharmaceutical company and/or one of its providers/intermediates or performed in part e.g., by a clinical laboratory or a different healthcare provider.

### Particular Embodiments

As will be apparent to those skilled in the art upon reading this description, each of the individual embodiments described and illustrated herein have discrete components and features which can be combined with the features of any of the other several embodiments without departing from the scope or spirit of the present invention. Particular combinations of the above embodiments detailed in different sections are described herein.

In an embodiment, the invention relates to a method of determining the risk of developing ADD in a subject, comprising:
a) determining in a sample of the subject comprising mitochondrial DNA, the methylation pattern in the D-loop region, and/or in the ND1 gene of the mitochondrial DNA, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6; and
b) combining the methylation pattern of one or more sites determined in step (a), with at least one clinical variable of the subject, as described herein;
wherein said combining is performed using a classification model for determining a risk score which correlates to the risk of developing ADD in the subject.

In an embodiment, the invention relates to a method of determining the risk of developing ADD in a subject, comprising:
a) determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6;
b) determining a risk score indicative of the risk of developing ADD, wherein the risk score is calculated using a classification model configured to combine the methylation pattern of one or more sites determined in step (a) with at least one clinical variable of the subject, as described herein.

In an embodiment, the invention relates to a method of determining the risk of developing ADD in a subject, comprising:
determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6;
wherein the methylation pattern is combined with at least one clinical variable of the subject, as described herein, to determine a risk score indicative of the risk of developing ADD using a classification model.

In an embodiment, the invention relates to a method of determining the risk of developing ADD in a subject, comprising:
determining a risk score indicative of the risk of developing ADD using a classification model configured to combine a methylation pattern with at least one clinical variable of the subject, as described herein;
wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6.

In an embodiment, the invention relates to a method of treating a subject having AD or at risk of developing ADD, comprising:
a) determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6;
b) determining a risk score indicative of the risk of developing ADD, wherein the risk score is calculated using a classification model configured to combine the methylation pattern of one or more sites determined in step (a) with at least one clinical variable of the subject, as described herein; and,
c) administering a treatment to the subject if the risk score indicates that the subject is at the risk of developing ADD.

In an embodiment, the invention relates to a method of treating a subject having AD or at risk of developing ADD, comprising:
a) determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6;
wherein the methylation pattern is combined with at least one clinical variable of the subject, as described herein, to determine a risk score indicative of the risk of developing ADD using a classification model; and,
b) administering a treatment to the subject if the risk score indicates that the subject is at the risk of developing ADD.

In an embodiment, the invention relates to a method of treating a subject having AD or at risk of developing ADD, comprising:
a) determining a risk score indicative of the risk of developing ADD using a classification model configured to combine a methylation pattern with at least one clinical variable of the subject, as described herein;
wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6; and,
b) administering a treatment to the subject if the risk score indicates that the subject is at the risk of developing ADD.

In an embodiment, the invention relates to a method of treating a subject having AD or at risk of developing ADD, comprising:
administering a treatment to the subject if a risk score indicates that the subject is at the risk of developing ADD,
wherein the risk score indicative of the risk of developing ADD is calculated using a classification model configured to combine the methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6;
with at least one clinical variable of the subject, as described herein.

In an embodiment, the invention is related to a method for identifying a human subject at risk of developing ADD, comprising:
a) determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6; and,
b) determining a risk score indicative of the risk of developing ADD, wherein the risk score is calculated using a classification model configured to combine the methylation pattern of one or more sites determined in step (a) with at least one clinical variable of the subject, as described herein.

In an embodiment, the invention relates to a method for identifying a human subject at risk of developing ADD, comprising:
determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6;
wherein the methylation pattern is combined with at least one clinical variable of the subject, as described herein, to determine a risk score indicative of the risk of developing ADD using a classification model.

In an embodiment, the invention relates to a method for identifying a human subject at risk of developing ADD, comprising:
determining a risk score indicative of the risk of developing ADD using a classification model configured to combine a methylation pattern with at least one clinical variable of the subject, as described herein;
wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6.

In an embodiment, the invention relates to a method for selecting a human subject for a treatment (such as a prophylactic treatment) for AD, comprising:
a) determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6; and,
b) determining a risk score indicative of the risk of developing ADD, wherein the risk score is calculated using a classification model configured to combine the methylation pattern of one or more sites determined in step (a) with at least one clinical variable of the subject, as described herein.

In an embodiment, the invention relates to a method for selecting a human subject for a treatment (such as a prophylactic treatment) for AD, comprising:
determining a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6;
wherein the methylation pattern is combined with at least one clinical variable of the subject, as described herein, to determine a risk score indicative of the risk of developing ADD using a classification model.

In an embodiment, the invention relates to a method for selecting a human subject for a treatment (such as a prophylactic treatment) for AD, comprising:
determining a risk score indicative of the risk of developing ADD using a classification model configured to combine a methylation pattern with at least one clinical variable of the subject, as described herein;
wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6.

Alternatively, another aspect of the invention relates to a combined biomarker for identifying a human subject at risk of developing ADD, wherein the combined biomarker comprises a classification model configured to combine a methylation pattern with at least one clinical variable of the subject, as described herein;
wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6.

In an embodiment, the invention relates to a classification model for identifying a human subject at risk of developing ADD configured to combine a methylation pattern with at least one clinical variable of the subject, as described herein;
wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6.

In an embodiment, the invention relates to a classification model for identifying a human subject at risk of developing ADD, wherein the model is configured to identify the human subject as being at risk of developing ADD, wherein the classification model is configured to combine a methylation pattern with at least one clinical variable of the subject, as described herein;
wherein the methylation pattern comprises a methylation pattern in (a) the D-loop region, and/or (b) the ND1 gene of mitochondrial DNA from a sample obtained from the subject, wherein the methylation pattern is determined in at least one site selected from the group consisting of:
   (i) the CpG sites in the D-loop region shown in Table 1,
   (ii) the CpG sites of the ND1 gene shown in Table 2,
   (iii) the CHG sites in the D-loop region shown in Table 3,
   (iv) the CHG sites in the ND1 gene shown in Table 4,
   (v) the CHH sites in the D-loop region shown in Table 5, and
   (vi) the CHH sites in the ND1 region shown in Table 6.

Particular embodiments described in the different sections of this description as e.g., about the classification model, the clinical variables, the sample, or the means for determining the methylation pattern, apply likewise to the above-described embodiments.

### EXAMPLES

### Example 1: Detection of mtDNA Methylation in Blood Samples

### 1.1 Materials and Methods

### 1) Blood Samples Collection

Human blood samples were collected in EDTA tubes to prevent blood coagulation. After obtention of samples in the laboratory, blood was directly processed for DNA extraction or aliquoted and stored at −80° C. until processed.

Samples from a total of 304 subjects were extracted, which were recruited from two different cohorts: Cohort A corresponds to the Australian Imaging, Biomarker & Lifestyle Flagship Study of Ageing (AIBL) cohort, and cohort B correspond to MCI patients recruited between 2015-2019 in Hospital de Bellvitge, Barcelona. These subjects were classified in three groups: controls (35.5%), MCI subjects which did not progress to ADD (30.6%) and MCI subjects which progressed to ADD (33.9%).

Control subjects were only available in cohort A, whereas MCI subjects were available in both cohorts.

### 2) Total DNA Extraction

Whole blood samples were processed to achieve total DNA extraction and copurification of both the genomic and mitochondrial DNA. DNA was isolated from human whole blood samples using the Wizard® Genomic DNA Purification Kit (#A1620, Promega), according to the manufacturer's instructions. Alternatively, samples were processed with the Maxwell® RSC Instrument that provided an easy method for efficient, automated purification of DNA from samples. DNA sample capture, washing and purification was done using paramagnetic beads. The Maxwell® RSC Blood DNA Kit (#AS1400, Promega) was used following manufacturer's specifications. The quality and quantity of purified DNA was determined with NanoDrop™ One Spectrophotometer from Thermo Fisher Scientific.

### 3) Bisulfite Treatment

Bisulfite conversion consists in the deamination of unmodified cytosines to uracil, leaving intact the modified bases 5-mC, i.e., methylated cytosines. Samples of total DNA (300 ng) were treated with bisulfite reagent using EZ DNA Methylation Kit (#D5001, Zymo Research), according to the manufacturer's protocol. In order to obtain a better bisulfite conversion, the incubation conditions of the step 2 of the protocol, which consisted in 15 min of incubation at 37° C., were substituted for 30 min of incubation at 42° C., as indicated in Appendix 1.A of the manufacturer's protocol. These last conditions are recommended to minimize an incomplete C to T Conversion. The treated DNA was finally resuspended in 30 μL of Nuclease free water.

### 4) Amplicon Library Preparation

The workflow for amplicon library construction was based on the Illumina “16S Metagenomic Sequencing Library Preparation Protocol”, which can be used to sequence regions of the 16S rRNA gene and other targeted amplicon sequences of interest. The amplicon library preparation allowed the obtention of mtDNA amplicons of interest and their preparation for Illumina MiSeq System processing.

### 4.1) First PCR: Amplicon PCR

The mtDNA regions of interest were amplified by PCR with specific degenerated primers (see Section 1.2.1 of the results), corresponding to sequences SEQ ID NO: 1-4, further containing overhanged Illumina adapters. When designing the primers for the region of interest, an overhang adapter sequence had to be added to the locus-specific primer for the region to be targeted, as indicated by Illumina's protocol.

The Illumina® overhang adapter sequences to be added to locus-specific sequences are (SEQ ID NO: 5, 6):

Forward overhang:
5′ TCGTCGGCAGCGTCAGATGTGTATAAGAGACAG-[locus-
specific sequence]
Reverse overhang:
5′ GTCTCGTGGGCTCGGAGATGTGTATAAGAGACAG-[locus-
specific sequence]

Amplification of the bisulfite converted DNA was performed using The FastStart™ High Fidelity PCR System (#3553400001, Roche). The final PCR mixture (25 μL) contained: 5 μL of Bisulfited DNA, 1×FastStart Buffer #2; 0.05 U FastStart HiFi Polymerase; 0.8 mM total dNTP (0.2 mM each dNTP); and 0.4 UM each forward and reverse primers. The reaction for the ND1 amplicon also included 5% of DMSO. Final volume was adjusted with nuclease free water.

Amplifications were performed in a SimpliAmp™ Thermal Cycler from Applied Biosystems.

In order to evaluate the resulting products of the first PCR, 3 L of each PCR product were analyzed by electrophoresis on 1.5% agarose gel stained with SybrSafe™ DNA Gel Stain (#S33102, Thermo Fisher Scientific), to verify the presence of the bands from the amplicons.

### 4.2) Clean-Up of the First PCR

AMPure XP beads (#A63881, Beckman Coulter) were used to purify the amplicons and separate them from free primers and primer dimer species. Steps were performed according to Illumina “16S Metagenomic Sequencing Library Preparation Protocol”. A ratio of 0.8× of AMpure Beads was used to purify the PCR amplicon product. Bead elution was performed in 14 μL of Buffer EB (#19086, Qiagen) and 12 μL were recovered from the beads.

### 4.3) Second PCR: Index PCR

Index PCR was performed to attach unique dual indices (UDI) and sequencing adapters from Illumina. PCR Index reaction was performed in a 50 μL reaction containing: 5 μL of DNA purified in the first PCR Clean-Up, 25 μL of KAPA HiFi HotStart Ready Mix (2x) (#7958935001, Roche), 10 μL of Unic dual indexes from Illumina, and 10 μL of Nuclease free water. It was performed using a SimpliAmp™ Thermal Cycler from Applied Biosystems™

### 4.4) Clean-Up of the Second PCR

Second PCR Clean-Up was performed using AMPure XP beads to clean up the final library before quantification. The 50 μL of the second PCR reaction were purified following the steps described in the “16S Metagenomic Sequencing Library Preparation Protocol” from Illumina. A ratio of 1.12× of AMPure XP beads was used, and a final elution was performed in 27.5 μL of Buffer EB and 25 μL were recovered from the beads.

### 4.5) Library Quantification, Normalization and Pooling

Quantification of the libraries was performed using a fluorometric quantification method that used dsDNA binding dyes with a Qubit® 3.0 Fluorometer, from Thermo Fisher Scientific. Quantification was performed with the Qubit™ dsDNA HS Assay Kit following manufacturer's instructions. After obtaining the Qubit quantification values in ng/μL, DNA concentration was calculated in nM, based on the size of DNA amplicons as determined by an Agilent Technologies 2100 Bioanalyzer trace with the following formula:

(Concentration in ng/μL)/(660 g/mol×average library size)×10=concentration in nM

To perform normalization, the final libraries were diluted using Buffer EB to 10 nM and a final 4 nM Pool of the amplicon libraries was prepared with Buffer EB in 20 μL of final volume.

In parallel from a 10 nM PhiX library (#FC-110-3001, Illumina), a 4 nM PhiX library dilution was prepared with Buffer EB in a 5 L of final volume (each run had to include a minimum of 5% PhiX to serve as an internal control for these low-diversity libraries).

For the preparation of cluster generation and sequencing, pooled amplicon libraries were denatured with NaOH and diluted with HT1 Buffer as follows: 5 μL of the 4 nM amplicon library and 5 μL of 0.2 N NaOH (freshly prepared) were introduced in a microcentrifuge tube, mixed briefly using a vortex, and centrifuged at 280×g at 20° C. for 1 minute. An incubation of 5 minutes at room temperature was performed to denature the DNA, and 990 μL of pre-chilled HT1 Buffer were added to the 10 μL of denatured DNA. Finally, HT1 results were added in a 20 pM denatured amplicon library in 1 mM NaOH. The denatured DNA was placed on ice until proceeding to final dilution.

The same steps were repeated for the 5 μL of 4 nM PhiX library to denature and dilute PhiX to result in a 20 pM PhiX denatured library.

A 7 pM denatured amplicon library was prepared mixing 210 μL of the 20 pM denatured amplicon library with 390 μL of pre-chilled HT1 Buffer in a final volume of 600 μL. Afterwards, a 10 pM denatured PhiX library was prepared mixing 300 μL of the 20 pM denatured PhiX library with 300 μL of pre-chilled HT1 Buffer in a final volume of 600 μL.

At the end a 25% of denatured PhiX library and 75% of denatured amplicon library were combined into a final volume of 600 L (150 μL from the 7 pM denatured amplicon library were discarded and were substituted by 150 μL of the 10 pM denatured PhiX library).

The combined amplicon library and PhiX control were set aside on ice until being ready to heat denature. The heat denaturation step was performed immediately before loading the library into the MiSeq reagent cartridge to ensure efficient template loading on the MiSeq flow cell.

Using a heat block, the combined library and PhiX control tube were incubated at 96° C. for 2 minutes. After the incubation, the tube was inverted 1-2 times to mix and immediately placed on ice. The tube was kept on ice for 5 minutes.

### 5) Template Loading and Run Setting on MiSeq Instrument

The Sequencing on MiSeq Instrument Using paired 300-bp reads was prepared, along with the MiSeq reagent Kit v3 (#MS-102-3003, Illumina).

When the Illumina v3 reagent cartridge was fully thawed and ready for use, prepared libraries were loaded onto the cartridge and run was set on MiSeq instrument following manufacturer's instructions.

### 6) Calculation of Mitochondrial Methylation Percentages

The percentage (%) of methylation of each cytosine site is calculated by means of the beta value (β). The β value is the ratio of methylated reads per site and the overall sum of methylated and unmethylated reads per site; that is:

$${β}_{i} = M / ( M + U )$$

where M is the number of methylated reads in site (i) and U is the number of unmethylated reads in the same site (i).

β values are between 0 and 1 with 0 being completely unmethylated and 1 fully methylated.

The percent of methylation (%) of each site is obtained by multiplying *100 the β value; that is:

$${\%}_{i} = {β}_{i} * 100$$

### 7) Differential Methylation Analysis

The analysis to compare methylation levels between in each methylation site was conducted using DSS (Dispersion Shrinkage for Sequencing data) Bioconductor package. This package is intended to identify Differentially Methylated Loci/Sites (DML/DMS) on bisulfite sequencing (BS-seq) data. A Bayesian hierarchical model was performed to estimate and shrink for each context site-specific dispersions, and a Wald test for Beta-Binomial distributions was applied to each of these contexts site-specific to test the null hypothesis that there is no differential methylation between the groups of samples. The model is set with one single main effect. Furthermore, in case of having technical biases due to batch effects or some sort of technical variability, these unwanted effects are considered in the model.

Raw p-values were adjusted for multiple testing using both False Discovery Rate (FDR) and a Family Wise Error Rate (FWER) approach. Any site with an adjusted p-value lower than 0.05 was considered as differentially methylated.

### 1.2 Results

1.2.1 Optimization of mtDNA Methylation Detection

The primers used herein (mentioned in section 4-Amplicon library preparation) were designed for an optimized detection of mtDNA methylation. In order to avoid a bias derived from the general assumption in the art that non-CpG cytosines are mainly unmethylated, inventors herein designed primers which included the least number of cytosines. Furthermore, the primers were degenerated to cover all possible methylated and no methylated scenarios due to the uncertain C/U conversion of the few cytosines residues included in the sequences. These primers were a mixture of oligonucleotide sequences which contain several possible nucleotide bases at certain position. Consequently, the probability to detect mitochondrial methylation were higher.

Consequently, the forward degenerated primers include Y, which refers to either C or T(Y=C/T), in any position wherein the reference sequence is a C. Further, as known in the art, reverse primers do not correspond to the reference sequence, but to the reversed complementary of the reference sequence. Therefore, the reverse primers do not include C sites of the reference sequence, but their complementary G sites. Thus, reverse degenerated primers will include R, which refers to either G or A (R=A/G), in any position wherein the reference sequence is a C, or wherein the complementary sequence includes a G. The four degenerated primers are:

D-loop region:
Forward Primer:
(SEQ ID NO: 1)
YAYTTGGGGGTAGYTAAAGTGAAYTG
Reverse primer:
(SEQ ID NO: 2)
TCCTACAARCATTAATTAATTAACACAC
ND1 gene:
Forward Primer:
(SEQ ID NO: 3)
ATAAAAYTTAAAAYTTTAYAGTYAGAG
Reverse primer:
(SEQ ID NO: 4)
TTRARTTTRATRCTCACCCTRATCA

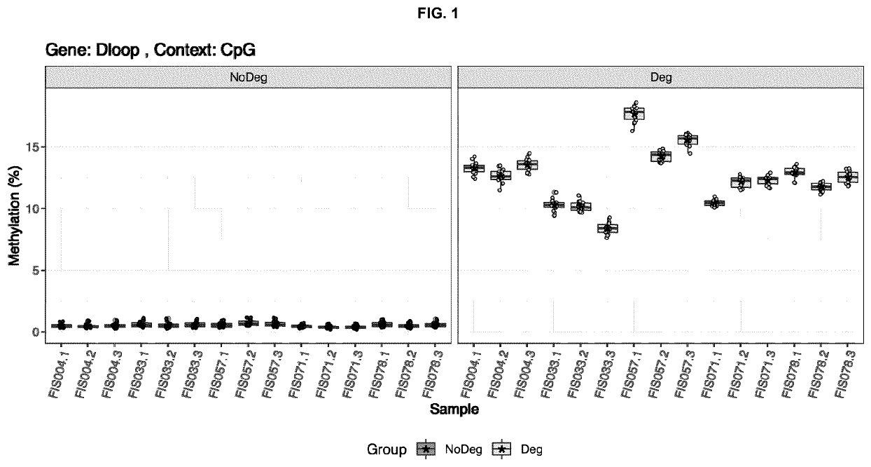

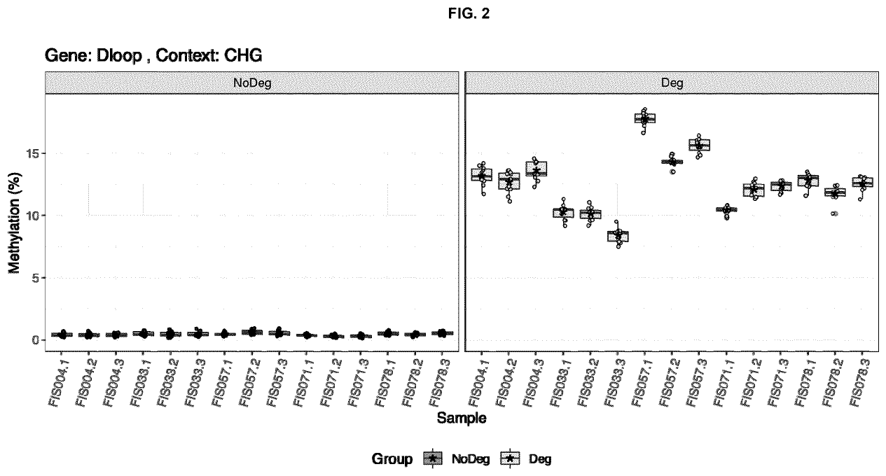

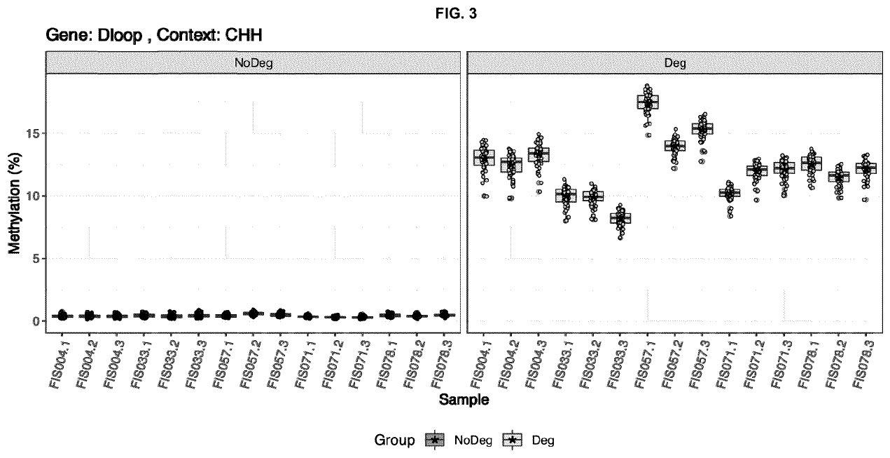

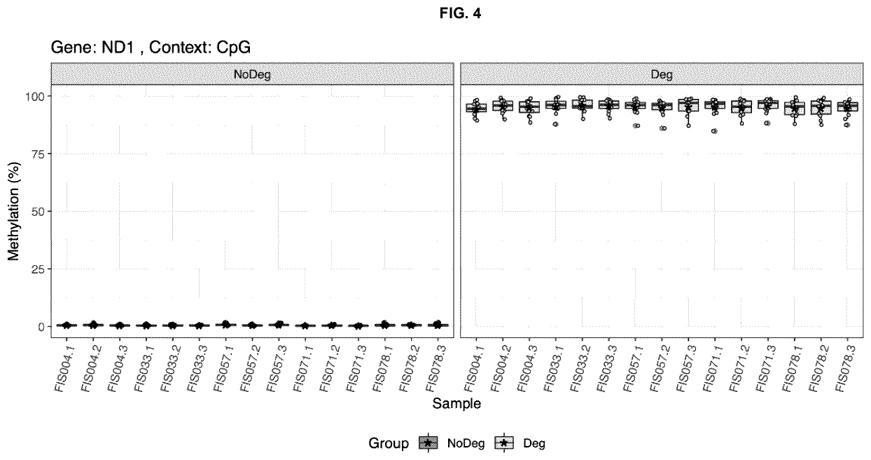

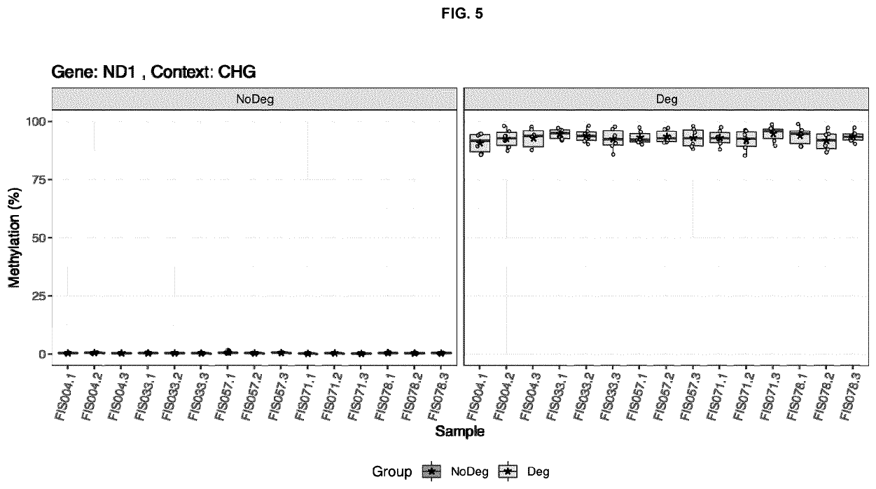

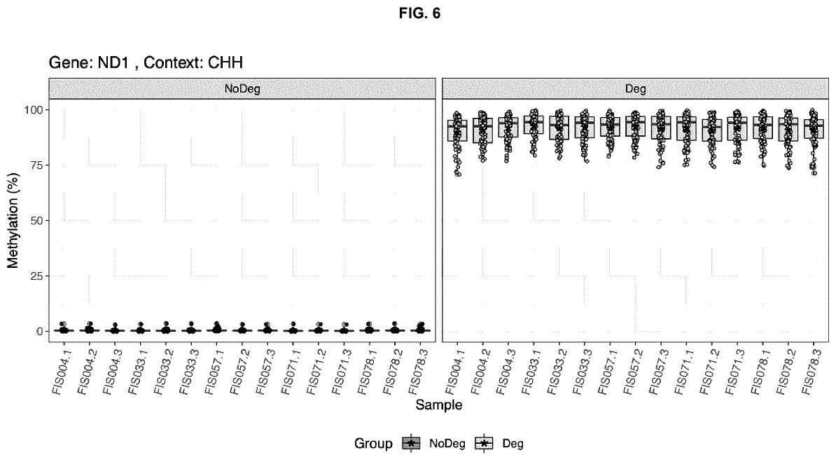

As shown in FIG. 1-6, the use of degenerated primers resulted in an extremely higher sensibility for the detection of mtDNA methylation, for both regions (i.e., D-loop region and ND1 gene) and in all three contexts (i.e., CpG, CHG, CHH). These results are certainly unexpected and exceed any anticipation of a higher methylation detection.

### 1.2.2 Mitochondrial DNA Methylation Patterns

The comparison of methylation levels between groups in terms of different contexts and regions resulted in a high number of significant differentially methylated comparisons.

### Example 2: Development of the classification model

A model was developed to classify subjects diagnosed with Mild Cognitive Impairment (i.e., CDR=0.5) susceptible of progressing to Alzheimer's Disease (ADD). Individual data of almost two hundred subjects, containing both clinical data and mitochondrial methylation information, was introduced in the model. Data regarding most of the patients was used to train the model, and the rest was used to evaluate the performance of the developed trained model. The trained model was shown capable to calculate the risk of a subject of developing ADD, and consequently classify said patient in the corresponding category between non-progression to ADD and progression to ADD, according to the performance evaluation.

### 2.1 Materials and Methods:

The classification model of the present invention was developed through the processing of individual data (clinical data and mitochondrial methylation measures generated in-house as described in EXAMPLE 1).

### 1) Target Data

After excluding subjects with missing values, a total of 199 subjects recruited from the two cohorts described in Section 1.1 of EXAMPLE 1 (Cohort A: 133 subjects, Cohort B: 66 subjects). These subjects were classified in three groups:
Controls: 79 subjects (39.7%), characterized in having:
   Clinical Dementia Rating (CDR) score of 0
   Clinical follow-up longer than 10 years.
ADD Non-progressed: 47 subjects (23.6%), characterized in having:
   Clinical Dementia Rating (CDR) score of 0.5 (related to having an early stage of MCI)
   Clinical follow-up longer than 36 months without showing progression of symptoms.
ADD Progressed: 73 subjects (36.7%), characterized in having:
   Clinical Dementia Rating which has progressed from 0.5 (MCI) to 1 (ADD).

Two main types of sources of information were covered in the analysis: clinical variables and methylation measures generated in-house (as described in EXAMPLE 1).

### 1.1) Clinical Variables

The prototype described herein includes the examination of 7 clinical variables:
Dementia stage *: corresponds to CDR global score, wherein Controls are CDR=0, ADD non-progressed are CDR=0.5 and ADD progressed are CDR=1 or higher.
*Dementia stage is used as the known correct output variable to train the model.
Sex: Female and Male.
Age: years of the subject.
APOE: genotype levels of Apolipoprotein E, i.e., E2.E2, E2.E3, E2.E4, E3.E3, E3.E4 and E4.E4.
alE4: recategorization of APOE genotype levels into 0 (E2.E2, E2.E3, and E3.E3), 1 (E2.E4 and E3.E4) and 2 (E4.E4).
SOB: Sum of Boxes Score is a score ranging from 0 to 18 obtained by summing each of the domain box scores described above for the calculation of CDR global score.
MMSE: Mini-Mental State Exam. Evaluation of five main items: orientation, fixation, concentration and calculation, memory and language, and construction with an output score between 1 and 30.

### 1.2) Methylation Measures

More than 200 variables were considered, each of them gathering the percentage of methylation for a single specific cytosine site, as described in EXAMPLE 1. Cytosine sites of three different contexts were considered: CpG, CHG and CHH, contained in one of the two loci: D-loop region and ND1 gene.

### 2) Exploratory Data Analysis

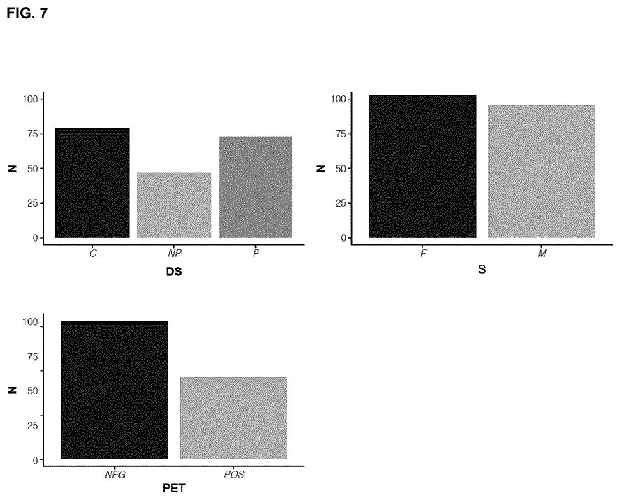

The Exploratory Data Analysis (EDA) was intended to describe every variable examined in the analysis. Sex showed a balanced number of subjects 103 (51.8%) females and 96 (48.2%) males. Table 7 shows frequency tables describing the categorical clinical variables: Dementia Stage, Cohort, APOE and alE4. FIG. 7 shows the bar diagrams of dementia stage, sex and alE4 variables.

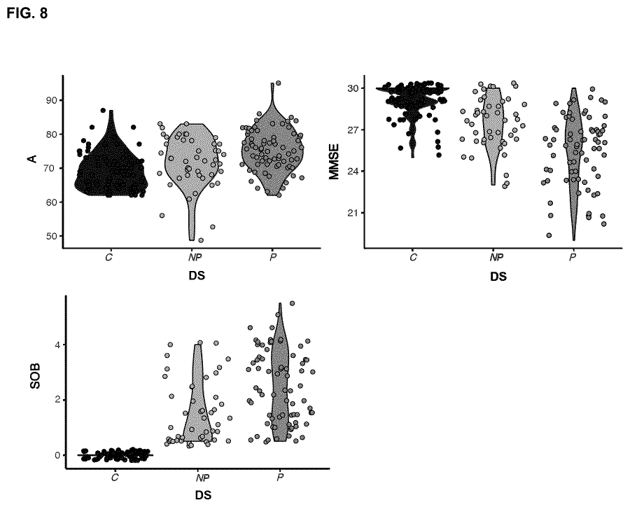

In addition, recruited individuals were 72.06±6.7 years old. They showed a 1.27±1.46 of SOB and a 27.37±2.50 in the MMSE. FIG. 8 shows the violin plots for variables Age, MMSE and SOB. Notice that MMSE and SOB distributions of ADD Progressed patients are higher dispersed than ADD non-progressed patients.

TABLE 7
Frequency tables of the categorical variables:
dementia stage, cohort, APOE and aIE4.

| Dementia Stage | Subjects (N) | Frequency |
|---|---|---|
| Control | 79 | 39.7% |
| ADD Non-Progressed | 47 | 23.6% |
| ADD Progressed | 73 | 36.7% |
| Cohort | | |
| AIBL | 133 | 66.8% |
| Admit | 66 | 33.2% |
| APOE | | |
| E2.E2 | 2 | 1.0% |
| E2.E3 | 23 | 11.6% |
| E2.E4 | 2 | 1.0% |
| E3.E3 | 102 | 51.3% |
| E3.E4 | 61 | 30.7% |
| E4.E4 | 9 | 4.5% |
| aIE4 | | |
| 0 | 127 | 63.8% |
| 1 | 63 | 31.7% |
| 2 | 9 | 4.5% |
| TOTAL | 199 | 100.0% |

### 3) Data Pre-Processing

This step was conducted to ensure and enhance the performance of the model training process. It consisted of creating dummy variables, removing zero- and near zero-variance variables, identifying and removing correlated variables, splitting the data into a training and testing data sets, centering and scaling both data sets, examining and visualizing the training data set. With regard the identification and removing correlated variables process, a pairwise correlation analysis based on the Pearson's correlation coefficient was performed. For those pairs showing high levels of absolute correlation values (>0.65), the variable with the largest mean absolute correlation was removed from the data set. In this regard, more than 2900 pairs of variables were identified to have an absolute correlation higher than 0.65. In addition, to study the relationship among individuals described by the set of quantitative and qualitative variables structured in groups of clinical and molecular data a Multiple Factor Analysis (MFA) was carried out. However, the MFA-derived features were not involved to build the classifier. The original corrected data was randomly split into the two main subsets; one for performing the model training (80% of the samples) and other for testing the classification model (20% of the samples). The random sampling process was driven within each class to preserve the overall class distribution of the data.

The training dataset included inputs and correct outputs, which allowed the model to learn over time. The correct output referred to the dementia stage of the subject (i.e., control, ADD progressed and ADD non-progressed), and the input data included data regarding methylation patterns and all remaining clinical variables.

### 4) Training of the Classification Model

Several supervised learning methods were considered to build the classification model according to their ability to process data with certain characteristics. In those lines, all selected methods were supervised classification methods capable of processing continuous and categorical data. These methods included: Linear Discriminant Analysis (LDA), Classification and Regression Trees (CART), k-Nearest Neighbors (kNN), Naive Bayes (NB), Support Vector Machines (SVM) with a linear kernel, Random Forest (RF), and Neural Network (NNET).

All methods were run using a 15-fold Cross-Validation, the total number of parameter combinations evaluated in SVM, RF and NNET was 5. NNET was estimated using a back-propagation approach iterating 1000 times.

### 5) Evaluation of the Performance of the Classification Model

To measure the performance of the training model the following metrics were estimated:
Accuracy: the overall agreement rate averaged over cross-validation iterations.
Kappa: the Cohen's unweighted Kappa statistic averaged across the resampling results.
Other common metrics: Sensitivity, Specificity, Positive Predicted Values, Negative Predicted Values, Precision, Prevalence, F1 score, Detection Rate, and Detection Prevalence.

For each of these statistics mean, median, minimum, maximum, and the first and third quartiles were estimated. Further, to evaluate the performance of the classification predictions of the model, a confusion matrix was built to show a cross-tabulation of the observed and predicted classes. This table was accompanied by Accuracy and the Kappa statistic, as well as the rest of common metrics used to evaluate the classification model. Additionally, a ROC curve was built to measure the model performance in classifying ADD Progressed versus ADD Non-progressed subjects.

### 2.2 Results

### 2.2.1 Performance of the Trained Model

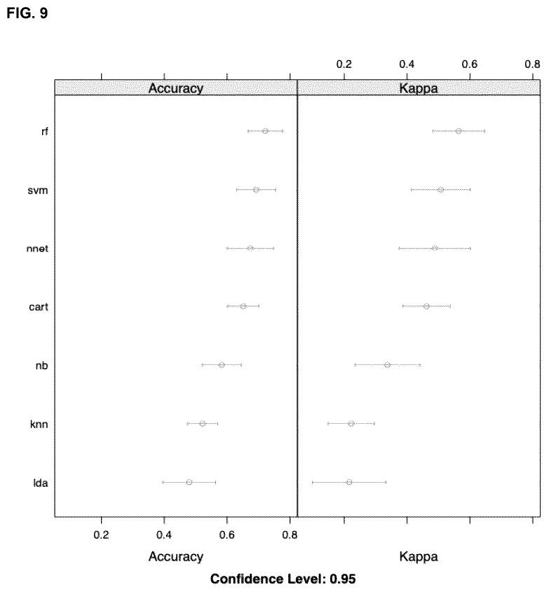

Overall, all classification models showed an adequate performance in terms of accuracy, with values over 0.5, except for LDA which has an accuracy of 0.48 (see FIG. 9). Particularly, models trained using RF, RVM and NNET and CART showed significantly high accuracy values over 0.65. Further, these four models also have significantly high Kappa values, ranging from 0.46 to 0.56. The best performing model was trained using Random Forest and showed outstanding performance values of 0.72 and 0.56 in terms of accuracy and Kappa, respectively. It is worth noting that random forest method is not only the best performing model but is also capable of better classifying borderline cases.

Consequently, the trained model based on random forest method was chosen to have the remaining testing data introduced, and therefore validate its performance, which had been firstly determined only based on training data.

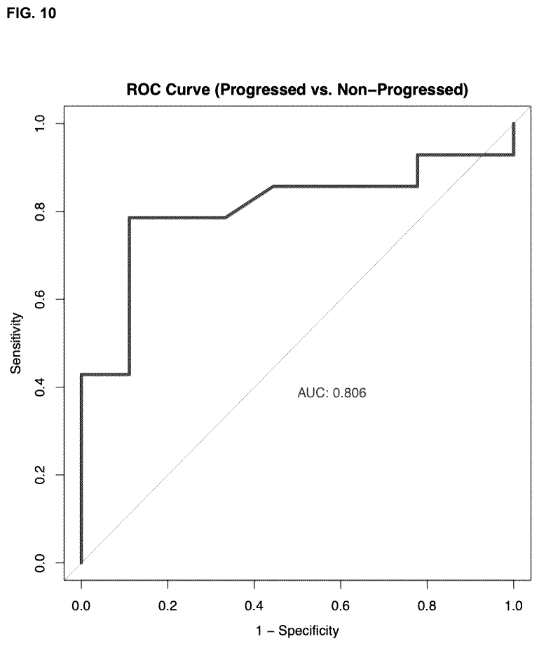

As a result, Random Forest model predictions on the testing data showed an overall accuracy score of 0.76 with a 95% Confidence Interval of 0.60 to 0.89, and a Kappa value of 0.63. Table 8 presents the confusion matrix, comparing model predictions with actual events in each group. Sensitivity and Specificity of the model to classify an MCI subject as ADD progressed were 0.86 and 0.7, respectively. The precision of the model was 0.63, and the F1 score was 0.72. FIG. 10 displays the ROC curve showing the performance of the classification model for the ADD progressed patients at all classification thresholds. In summary, the classification model developed herein showed a very high performance in the identification of progressed MCI.

TABLE 8
Confusion matrix

| | Reference | | |
|---|---|---|---|
| | | ADD Non- | ADD |
| Prediction | Control | progressed | Progressed |
| Control | 15 | 0 | 0 |
| ADD Non-progressed | 0 | 2 | 2 |
| ADD Progressed | 0 | 7 | 12 |

In those lines, Table 9 shows some examples of the predictions of the classification model, with an output value corresponding of the risk score of progressing to ADD.

TABLE 9
Predictions of the classification model of subjects included in the testing
data set. “Prob” refers to “probability”.

| | | Dementia | | Prob. | Prob.Non- | Prob. |
|---|---|---|---|---|---|---|
| Subject | Cohort | Stage | Prediction | Control | Progressed | Progressed |
| 1 | AIBL | Control | Control | 0.876 | 0.04 | 0.084 |
| 2 | AIBL | Control | Control | 0.988 | 0.004 | 0.008 |
| 3 | ADmit | Progressed | Progressed | 0.008 | 0.418 | 0.574 |
| 4 | AIBL | Progressed | Progressed | 0.024 | 0.226 | 0.75 |
| 5 | AIBL | Non-progressed | Non-progressed | 0.018 | 0.578 | 0.404 |
| 6 | ADmit | Non-progressed | Progressed | 0.03 | 0.434 | 0.536 |

### 2.2.2 Variables Importance

In regard to the importance of each variable introduced in the classification model, it describes the contribution to the determination of the risk to develop ADD of each clinical variable and methylation pattern of each site.

Firstly, it is worth noting that the model disclosed in EXAMPLE 2 did not include any clinical variable which may be obtained through invasive or highly costly techniques such as PET. In the current clinical practice, PET to detect beta-amyloid plaques is indeed considered a highly informative diagnostic technique for AD diagnosis. However, the classification model developed herein was clearly capable of predicting the risk of developing ADD with very high-performance indicators, despite not using such information.

In terms of variables importance, SOB score and MMSE were the two variables which contribute the most to the determination of a risk score of developing ADD. As stated above, these relate to a semi-structured interview and a mini-mental state exam, respectively. Further, age was the fourth variable with a higher importance. Thus, these results support the importance of combining clinical variables with variables relating to mitochondrial methylation to adequately determine the risk of developing ADD. Further, fifteen of the twenty variables with a highest importance rate corresponded to mitochondrial methylation data of CHH sites of the ND1 gene, whereas only two of these variables related to CHH sites in the D-loop region. This confirms the significant contribution of CHH sites of the ND1 gene in the determination of such risk.

Finally, variables related to polymorphism apolipoprotein E showed the lowest contribution to the determination of the risk score of progressing to ADD. These results appear surprising as polymorphisms in apolipoprotein E are a well-known major genetic risk determinant of late onset ADD, and the results herein suggest that said polymorphism might not be as significant for the detection of subjects at risk of developing ADD at early stages of dementia (i.e., MCI).

### EXAMPLE 3: Development of the Classification Model Including PET as a Clinical Variable

A model was developed to classify subjects diagnosed with Mild Cognitive Impairment (i.e., CDR=0.5) susceptible of progressing to Alzheimer's Disease dementia (ADD), as described in EXAMPLE 1, further including PET for detecting beta-amyloid plaques as a clinical variable.

In the current clinical practice, patients suspected to suffer from MCI or ADD, are performed an amyloid PET test which can detect beta-amyloid protein plaques in the brain. However, amyloid PET cannot distinguish between subjects diagnosed with MCI which will progress or those which will not progress to ADD. Thus, for those subjects where PET test had already been performed, inventors aimed to develop a classification model while taking advantage of data resulting from amyloid PET test (positive or negative).

As described in EXAMPLE 2, individual data of almost two hundred subjects, containing both clinical data and mitochondrial methylation information, was introduced in the model. Data regarding most of the patients was used to train the model, and the rest was used to evaluate the performance of the developed trained model. The trained model was shown capable to calculate the risk of a subject of developing ADD, and consequently classify said patient in the corresponding category between non-progression to ADD and progression to ADD, according to the performance evaluation.

All subjects included in EXAMPLE 2 were included in the present example, as there was data available for all of them regarding amyloid PET test.

### 3.1 Materials and Methods:

The classification model of the present invention was developed through the processing of individual data (clinical data and mitochondrial methylation measures generated in-house as described in EXAMPLE 1).

### 1) Target Data

Subjects included in the present experiment correspond to subjects included in EXAMPLE 2.

Again, two main types of sources of information were covered in the analysis: clinical variables and methylation measures generated in-house (as described in EXAMPLE 1).

In regard to the clinical variables, the prototype described herein includes ten variables, nine of which correspond to those clinical variables described in EXAMPLE 2. The tenth variable corresponds to PET β-amyloid: Positron Emission Tomography measurement to detect levels of amyloid protein aggregates in the brain: POS (positive) or NEG (negative).

### 1.2) Methylation Measures

More than 200 variables, each of them gathering the percentage of methylation for a single specific cytosine site. Cytosine sites of three different contexts were considered: CpG, CHG and CHH, contained in any of the two genes D-loop and ND1 gene.

### 2) Exploratory Data Analysis

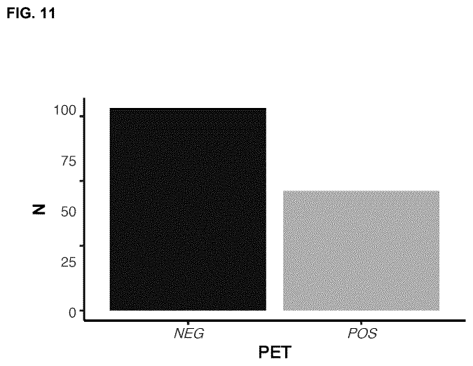

The Exploratory Data Analysis (EDA) was performed as described in the corresponding section of EXAMPLE 2. As described above, subjects of EXAMPLE 3 are the same subjects as EXAMPLE 2. Therefore, results of EDA can be found in FIG. 7-8 and Table 7. Additionally, Table 10 and FIG. 11 show the frequency of positive and negative results of the β-amyloid PET.

TABLE 10
Frequency table of PET.

| PET | Subjects (N) | Frequency |
|---|---|---|
| Negative | 125 | 62.8% |
| Positive | 74 | 74.2% |
| Total | 199 | 100.0% |

### 3) Data Pre-Processing

As described in EXAMPLE 2, this step was conducted to ensure and enhance the performance of the model training process. This comprises splitting the data into a training and testing data sets and examining and visualizing the training data set.

### 4) Training of the Classification Model

As described in EXAMPLE 2, several supervised learning methods were considered to build the classification model according to their ability to process data with certain characteristics. In those lines, all selected methods were supervised classification methods capable of processing continuous and categorical data. These methods included: Linear Discriminant Analysis LDA), Classification and Regression Trees (CART), k-Nearest Neighbors (kNN), Naive Bayes (NB), Support Vector Machines (SVM) with a linear kernel, Random Forest (RF), and Neural Network (NNET).

All methods were run using a 15-fold Cross-Validation, the total number of parameter combinations evaluated in SVM, RF and NNET was 5. NNET was estimated using a back-propagation approach iterating 1000 times.

### 5) Evaluation of the Performance of the Classification Model

The evaluation of the performance of the classification model was performed as described in the corresponding section of EXAMPLE 2.

### 3.2 Results

### 3.2.1. Performance of the Trained Model

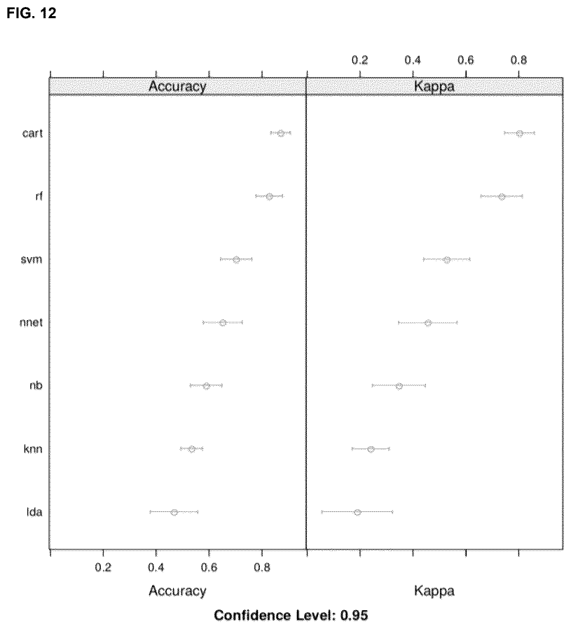

Overall, all training model showed an adequate performance and most of them had significantly high-performance values, as shown in FIG. 12. In terms of accuracy all classification models showed values over 0.5, except for the LDA model, with an accuracy of 0.47. Kappa values were lower and only CART, SVM and RF had values over 0.5.

Results of the classification model training indicates that the best performance seems to be a CART model, with an average accuracy of 0.87 and the Kappa value of 0.80. These results clearly show that the training model developed using CART method would be a good model for classifying MCI patients.

However, these types of models may be sensitive to outlier observations, and consequently misclassify subjects.

Further, the trained classification model resulting from using the random forest method also showed very good performance values, with a mean accuracy value of 0.83 and a Kappa value of 0.73. It is worth noting that random forest method is capable of better classifying of borderline cases. Consequently, the trained model based on random forest method was chosen to have the remaining testing data introduced, and therefore validate its performance, which had been firstly determined only based on training data.

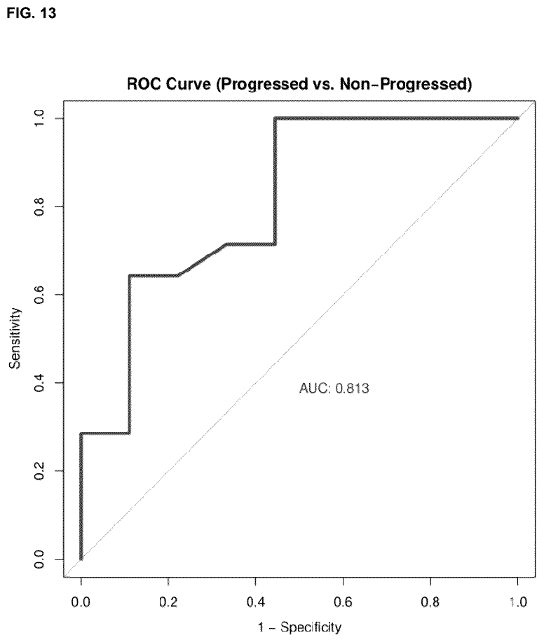

As a result, Random Forest model predictions on the testing data showed an overall accuracy score of 0.89 with a 95% Confidence Interval of 0.75 to 0.97, and a Kappa value of 0.84. Table 11 presents the confusion matrix, comparing model predictions with actual events in each group. Sensitivity and Specificity of the model to classify an MCI subject as ADD progressed are 1 and 0.83, respectively. The precision of the model is 0.78, and the F1 score is 0.86. FIG. 13 displays the ROC curve showing the performance of the classification model for the ADD progressed patients at all classification thresholds. In summary, the classification model developed herein shows a very high performance in the identification of ADD progressed.

TABLE 11
Confusion matrix

| | Reference | | |
|---|---|---|---|
| | | ADD Non- | ADD |
| Prediction | Control | progressed | Progressed |
| Control | 15 | 0 | 0 |
| ADD Non-progressed | 0 | 5 | 0 |
| ADD Progressed | 0 | 4 | 14 |

In those lines, Table 11 shows some examples of the predictions of the classification model, with an output value corresponding of the risk score of progressing to ADD.

TABLE 12
Predictions of the classification model of subjects included in the testing data set.

| | | | | Prob. | Prob. Non- | Prob. |
|---|---|---|---|---|---|---|
| Subject | Cohort | Dementia Stage | Prediction | Control | Progressed | Progressed |
| 1 | AIBL | Control | Control | 1.00 | 0.00 | 0.00 |
| 2 | AIBL | Control | Control | 1.00 | 0.00 | 0.00 |
| 3 | ADmit | Progressed | Progressed | 0.00 | 0.108 | 0.892 |
| 4 | AIBL | Progressed | Progressed | 0.00 | 0.044 | 0.956 |
| 5 | AIBL | Non-progressed | Progressed | 0.00 | 0.266 | 0.734 |
| 6 | ADmit | Non-progressed | Progressed | 0.00 | 0.228 | 0.772 |

### 3.2.2 Variables Importance

As described in EXAMPLE 2, the importance of each variable introduced in the classification model describes the contribution to the determination of the risk to develop ADD.

In the classification model of this Example, again SOB score and MMSE were two variables among the highest contributors to the determination of a risk score of developing ADD: first and third, respectively. However, in this classification model, a positive PET result contributed highly to such determination, being the second most determining variable.

As in EXAMPLE 2, most of the variables with a highest importance rate corresponded to mitochondrial methylation data of CHH sites of the ND1 gene. This confirmed again the significant contribution of CHH sites of the ND1 gene in the determination of such risk.

Finally, and again as in EXAMPLE 2, variables related to polymorphism apolipoprotein E showed the lowest contribution to the determination of the risk score of progressing to ADD.

### EXAMPLE 4: Development of the Classification Model Including Only PET as a Clinical Variable

A model was developed to classify subjects diagnosed with Mild Cognitive Impairment (i.e., CDR=0.5) susceptible of progressing to ADD. Individual data of 211 subjects, containing both clinical data (including only data regarding PET test) and mitochondrial methylation information, was introduced in the model. Data regarding most of the patients was used to train the model, and the rest was used to evaluate the performance of the developed trained model. The trained model was shown capable to calculate the risk of a subject of developing ADD, and consequently classify said patient in the corresponding category between non-progression to ADD and progression to ADD, according to the performance evaluation.

In this Example, the number of subjects used to train and test the model is higher than in previous Examples (from 199 to 211 subjects), as a result of a higher availability of samples and information. Noticeably, such higher number of subjects allows for the development of a more reliable classification model. Thus, including a higher number of subjects into a classification model as described herein, it is expected to achieve even higher performances.

### 4.1 Materials and Methods:

The classification model of the present invention was developed through the processing of individual data (clinical data (PET test) and mitochondrial methylation measures).

### 1) Target Data

After excluding subjects with missing values, a total of 211 subjects recruited from three cohorts (A, B and C). Cohort A corresponds to the Australian Imaging, Biomarker & Lifestyle Flagship Study of Ageing (AIBL) cohort (129 subjects), cohort B correspond to MCI patients recruited between 2015-2019 in Hospital de Bellvitge, Barcelona (74 subjects), and cohort C correspond to MCI patients recruited between 2014-2019 in Hospital Clínic de Barcelona (8 subjects). These subjects were classified in three groups:
Controls: 68 subjects (32.23%), characterized in having:
   Clinical Dementia Rating (CDR) score of 0.
   Clinical follow-up longer than 10 years.
ADD Non-progressed: 58 subjects (27.49%), characterized in having:
   Clinical Dementia Rating (CDR) score of 0.5 (related to having an early stage of MCI).
   Clinical follow-up longer than 36 months without showing progression of symptoms.
ADD Progressed: 85 subjects (40.28%), characterized in having:
   Clinical Dementia Rating which has progressed from 0.5 (MCI) to 1 (ADD).

Control subjects were only available in cohort A, whereas MCI subjects were available in all three cohorts.

Two main types of sources of information were covered in the analysis: clinical variables and methylation measures generated in-house (as described in EXAMPLE 1).

### 1.1) Clinical Variables

The prototype described herein includes the examination of 2 clinical variables:
Dementia stage *: corresponds to CDR global score, wherein Controls are CDR=0, ADD non-progressed are CDR=0.5 and ADD progressed are CDR=1 or higher.
*Dementia stage is used as the known correct output variable to train the model.
PET test (POS or NEG): corresponds to the amyloid PET test performed on subjects to detect the presence of beta-amyloid protein plaques in the brain.

As discussed in EXAMPLE 2, in the current clinical practice, patients suspected to suffer from MCI or ADD, are performed an amyloid PET test which can detect beta-amyloid protein plaques in the brain. However, amyloid PET cannot distinguish between subjects diagnosed with MCI which will progress or those which will not progress to ADD. Thus, for those subjects where PET test had already been performed, inventors aimed to develop a classification model which can use such data resulting from amyloid PET test (positive or negative).

### 1.2) Methylation Measures

More than 200 variables were considered, each of them gathering the percentage of methylation for a single specific cytosine site, as described in EXAMPLE 1. Cytosine sites of three different contexts were considered: CpG, CHG and CHH, contained in one of the two loci: D-loop region and ND1 gene.

### 2) Exploratory Data Analysis

The Exploratory Data Analysis (EDA) was intended to describe every variable examined in the analysis. Sex showed a balanced number of subjects 110 (52.13%) females and 101 (47.86%) males. Table 13 shows frequency tables describing the categorical clinical variables: Dementia Stage and PET.

TABLE 13
Frequency tables of the categorical
variables: dementia stage and PET.

| Dementia Stage | Subjects (N) | Frequency |
|---|---|---|
| Control | 68 | 32.23% |
| ADD Non- | 58 | 27.49% |
| Progressed | | |
| ADD Progressed | 85 | 40.28% |
| PET | | |
| Negative | 133 | 63.03% |
| Positive | 78 | 36.97% |
| TOTAL | 211 | 100.0% |

In addition, recruited individuals were 72.2±7 years old; particularly, 69.3±5.28 years old in the Control group, 71.22±7.67 years old in the ADD Non-progressed group, and 75.18±6.62 years old in the ADD Progressed group.

### 3) Data Pre-Processing

This step was conducted as in EXAMPLE 2, to ensure and enhance the performance of the model training process. This comprises splitting the data into a training and testing data sets, examining and visualizing the training data set.

The training dataset included inputs and correct outputs, which allowed the model to learn over time. The correct output referred to the dementia stage of the subject (i.e., control, ADD progressed and ADD non-progressed), and the input data included data regarding methylation patterns and PET test as clinical variable.

### 4) Training of the Classification Model

Several supervised learning methods were considered to build the classification model according to their ability to process data with certain characteristics. In those lines, all selected methods were supervised classification methods capable of processing continuous and categorical data. These methods included: Linear Discriminant Analysis (LDA), Penalized Multinomial Regression (PMR), Classification and Regression Trees (CART), k-Nearest Neighbors (kNN), Naive Bayes (NB), Support Vector Machines with Radial Basis Function kernel (SVM.Radial), Random Forest (RF), and Neural Network (NNET).

All methods were run using a 3 repeated 10-fold Cross-Validation, the total number of parameter combinations evaluated in SVM, RF and NNET was 5. NNET was estimated using a back-propagation approach iterating 1000 times.

### 5) Evaluation of the Performance of the Classification Model

To measure the performance of the training model the following metrics were estimated:
Accuracy: the overall agreement rate averaged over cross-validation iterations.
Kappa: the Cohen's unweighted Kappa statistic averaged across the resampling results.

For each of these statistics mean, median, minimum, maximum, and the first and third quartiles were estimated. To evaluate the performance of the classification predictions of the model, a confusion matrix was built to show a cross-tabulation of the observed and predicted classes. This table was accompanied by Accuracy and the Kappa statistic, as well as the common metrics used to evaluate classification models, such as Sensitivity, Specificity, Positive Predicted Values, Negative Predicted Values, Precision, Prevalence, F1 score, Detection Rate, and Detection Prevalence. Additionally, a ROC curve was built to measure the model performance in classifying ADD Progressed versus ADD Non-progressed subjects.

### 4.2 Results

### 4.2.1 Performance of the Trained Model

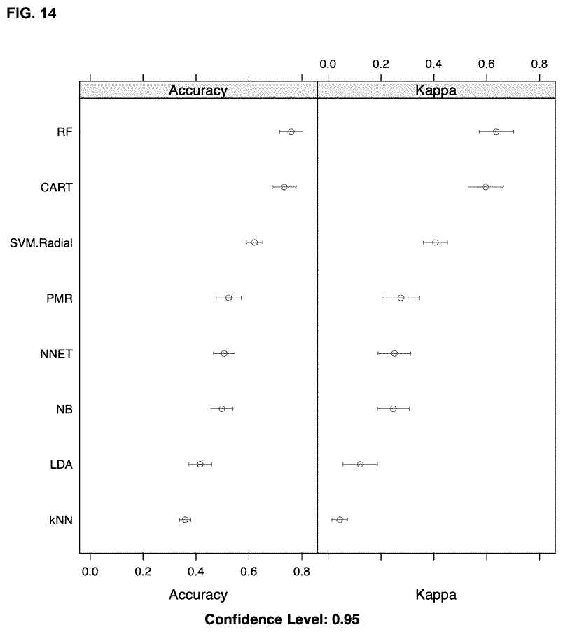

Overall, most classification models showed an adequate performance in terms of Accuracy, with values over 0.5 in the case of RF, CART, SVM. Radial, PMR and NNET (see FIG. 14).

Particularly, models trained using RF and CART showed significantly high Accuracy values over 0.73. Further, these two models also have high Kappa values over 0.59. The best performing model was trained using Random Forest and showed outstanding performance values of 0.76 and 0.636 in terms of Accuracy and Kappa, respectively. It is worth noting that Random Forest method is not only the best performing model but is also capable of better classifying borderline cases.

Consequently, the trained model based on Random Forest method was chosen to have the remaining testing data introduced, and therefore validate its performance, which had been firstly determined only based on training data.

As a result, Random Forest model predictions on the testing data showed an overall Accuracy score of 0.756 with a 95% Confidence Interval of 0.597 to 0.876, and a Kappa value of 0.63. Table 14 presents the confusion matrix comparing model predictions with actual events in each group.

TABLE 14
Confusion matrix

| | Reference | | |
|---|---|---|---|
| | | ADD Non- | ADD |
| Prediction | Control | progressed | Progressed |
| Control | 12 | 3 | 1 |
| ADD Non-progressed | 1 | 6 | 3 |
| ADD Progressed | 0 | 2 | 13 |

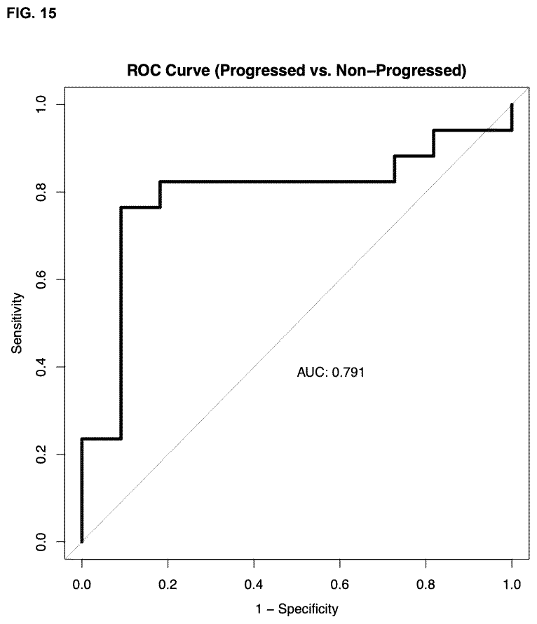

Sensitivity and Specificity of the model to classify an MCI subject as ADD progressed were 0.76 and 0.92, respectively. The precision of the model was 0.87, and the F1 score was 0.81. The Positive Predictive Value was 0.87, and the Negative predictive Value was 0.85. FIG. 15 displays the ROC curve showing the performance of the classification model for the ADD progressed patients at all classification thresholds, which has an AUC=0.791. In summary, the classification model developed herein showed a very high performance in the identification of progressed MCI.

In those lines, Table 15 shows some examples of the predictions of the classification model, with an output value corresponding of the risk score of progressing to ADD.

TABLE 15
Predictions of the classification model of subjects included in the testing
data set. “Prob” refers to “probability”.

| | | | | Prob. | Prob. Non- | Prob. |
|---|---|---|---|---|---|---|
| Subject | Cohort | Dementia Stage | Prediction | Control | Progressed | Progressed |
| 1 | AIBL | Control | Control | 0.808 | 0.152 | 0.040 |
| 2 | AIBL | Control | Control | 0.884 | 0.050 | 0.066 |
| 3 | ADmit | Progressed | Progressed | 0.010 | 0.126 | 0.864 |
| 4 | AIBL | Progressed | Progressed | 0.062 | 0.046 | 0.892 |
| 5 | ADmit | Non-progressed | Non-progressed | 0.170 | 0.592 | 0.238 |
| 6 | AIBL | Non-progressed | Progressed | 0.038 | 0.164 | 0.798 |

### 4.2.2 Variables Importance

As described in EXAMPLES 2 and 3, the importance of each variable introduced in the classification model describes the contribution to the determination of the risk to develop ADD.

In the classification model of this Example, only one clinical variable is considered for the determination of the risk to develop ADD: PET scan. In line with previous EXAMPLE 3, a positive PET result contributed highly to such determination, being the most determining variable.

Mitochondrial methylation data of two CHH sites of the D-loop region were the second and fourth most contributing variables, corresponding to a 33% and 8.3% contribution, respectively. Further, two CpG sites of the ND1 gene were the third and fifth most contributing variables, with a contribution of 12.2% and 7.6%, respectively. Further, most of the variables with a highest importance rate corresponded to mitochondrial methylation data of CHH sites of the ND1 gene, accounting for half of the twenty highest contributing variables.

Remarkably, 86 out of 142 variables contributed in over 1% to the determination of the risk of developing ADD, 35 out of 142 in over 2%, 7 out of 142 in over 5% and 3 out of 142 in over 10%.

This Example clearly shows that classification models which are developed including only one clinical variable can achieve a significantly high performance, demonstrating its capacity to identify subjects suffering from MCI which will develop ADD in later stages. In this case, PET scan was selected as the clinical variable to be included in the classification. However, it is not to be interpreted that PET scan is a compulsory clinical variable required in development of classification models to achieve outstanding results. As shown in EXAMPLE 2, the construction of models not including PET scan as a clinical variable also result also result in very high-performance rates.

4.2.3 Variables Importance. Differences Between Classification Models

In terms of variables regarding the mitochondrial methylation patterns of different sites, those variables contributing the most to the determination of the risk of developing ADD differ between EXAMPLES 2-4. This is because the contribution of each variable depends on several aspects, the number of variables considered in the model (e.g., which clinical variables are included), the number of subjects used in the training of the classification model (in order to avoid over/under-fitting issues), the customization process of the parameters during the training process of the model, etc.

In those lines, the variables included in each classification model will depend on the information available from each subject, and consequently the importance/contribution of each selected variable will vary among classification models. Thus, it is difficult to define an exact set of variables or a specific number of variables to be considered when constructing a classification model. Contrariwise, it is desirable to develop classification models with the ability to adapt to the information available and can be constructed using variables selected according to their importance/contribution in each particular situation, as demonstrated in the present examples. In other words, the variables included in each classification model will be defined according to their contribution during the training process, rather than arbitrarily constituting a set of predefined variables or a minimum number of variables that may not contribute as much in other scenarios. Whichever the case, the main objective is achieve an Accuracy value as higher as possible.

Finally, as stated above, the higher the number of subjects included in the training process, the more reliable this model can be. However, the acquisition of samples and clinical information of subjects is a complicated process for many reasons: suitable subjects are limited, and they must have been monitored for long periods of time; the accessibility to samples is limited and expensive; the quality of the samples can be compromised; the information on subjects include missing data, etc. Overall, the data available for the development of a classification model is not to be taken for granted as it is the result of a burdensome and difficult process.

1. A method of determining the risk of developing Alzheimer's disease dementia in a subject, comprising:
a) determining a methylation pattern in (a) the D-loop region, and/or (b) the ND1 gene of mitochondrial DNA from a sample obtained from the subject, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6; and
b) determining a risk score indicative of the risk of developing Alzheimer's disease dementia, wherein the risk score is calculated using a classification model configured to combine the methylation pattern of one or more sites determined in step (a) with at least one clinical variable of the subject selected from the group consisting of: sex, Sum of Boxes Score, Mini-Mental State Exam, Positron Emission Tomography, presence or absence of β-amyloid protein, age, genotype levels of Apolipoprotein E, and recategorized genotype levels of Apolipoprotein E.

2. The method according to claim 1, wherein the methylation pattern is determined using at least one oligonucleotide with a length between 15 and 100 nucleotides capable of specifically hybridizing with a mitochondrial DNA sequence comprising a methylation site selected from the group consisting of (i)-(vi) sequences, particularly the oligonucleotide comprises at least one sequence selected from the group consisting of SEQ ID NO: 1, SEQ ID NO: 2, SEQ ID NO: 3 and SEQ ID NO: 4.

3. The method according to claim 1, wherein determining the methylation pattern is determined by bisulfite sequencing.

4. The method according to claim 1, wherein the risk score is associated to progression to Alzheimer's disease dementia or to Non-progression to Alzheimer's disease dementia.

5. The method according to claim 1, wherein the methylation pattern is determined in at least all the CHH sites in the ND1 region shown in Table 6.

6. The method according to claim 1, wherein the methylation pattern is determined in all sites CpG, CHG and CHH sites of the D-loop region and ND1 gene.

7. The method according to claim 1, wherein the classification model is developed using a supervised machine learning method.

8. The method according to claim 7, wherein the supervised machine learning method is selected from the group consisting of Linear Discriminant Analysis (LDA), Penalized Multinomial Regression (PMR), Classification and Regression Trees (CART), k-Nearest Neighbors (kNN), Naive Bayes (NB), Support Vector Machines (SVM) with a linear kernel, Support Vector Machines with Radial Basis Function Kernel (SVM.Radial), Random Forest (RF) and Neural Network (NNET), and particularly is Random Forest (RF).

9. The method according to claim 7 wherein the classification model is trained with a training set comprising mitochondrial methylation patterns for each methylation site in a plurality of samples associated to a plurality of subjects and comprising clinical variables associated to a plurality of subjects, wherein each subject is assigned a Dementia Stage Classification selected from the group consisting of control, Alzheimer's disease dementia progressed and Alzheimer's disease dementia non-progressed.

10. The method according to claim 1, wherein determining the risk score comprises correlating each of at least one of the methylation patterns determined in step (a) and each of at least one clinical variable with their weight determined during the training of the classification model.

11. The method according to claim 1, wherein the subject is a human subject diagnosed with a Clinical Dementia Rating score of 0.5 corresponding to Mild Cognitive Impairment.

12. The method according to claim 1, wherein the sample is a biofluid selected from the group consisting of blood, plasma, saliva, cerebrospinal fluid, brain sample, skin sample and urine.

13. A kit comprising oligonucleotides with a length between 15 and 100 nucleotides, comprising the nucleic acid sequences SEQ ID NO: 1 and SEQ ID NO: 2 or the nucleic acid sequences SEQ ID NO: 3 and SEQ ID NO: 4.

14. The kit according to claim 13, comprising oligonucleotides with a length between and 100 nucleotides, comprising the nucleic acid sequences SEQ ID NO: 1, SEQ ID NO: 2, SEQ ID NO: 3 and SEQ ID NO: 4.

15. A computer-implemented method of determining the risk of developing Alzheimer's disease dementia in a subject, comprising:
a) receiving data relating to a methylation pattern in (a) the D-loop region, and/or (b) the ND1 gene of mitochondrial DNA of the subject, wherein the methylation pattern is determined in at least one site selected from the group consisting of:
(i) the CpG sites in the D-loop region shown in Table 1,
(ii) the CpG sites of the ND1 gene shown in Table 2,
(iii) the CHG sites in the D-loop region shown in Table 3,
(iv) the CHG sites in the ND1 gene shown in Table 4,
(v) the CHH sites in the D-loop region shown in Table 5, and
(vi) the CHH sites in the ND1 region shown in Table 6; and
b) determining a risk score indicative of the risk of developing Alzheimer's disease dementia, wherein the risk score is calculated using a classification model configured to combine the methylation pattern of one or more sites determined in step (a) with at least one clinical variable of the subject selected from the group consisting of: sex, Sum of Boxes Score, Mini-Mental State Exam, Positron Emission Tomography, presence or absence of β-amyloid protein, age, genotype levels of Apolipoprotein E, and recategorized genotype levels of Apolipoprotein E.
